(19) United States
(12) Patent Application Publication
(10) Pub. No.: US 2012/0130729 A1
(43) Pub. Date: May 24, 2012

(54) SYSTEMS AND METHODS FOR EVALUATION OF EXAM RECORD UPDATES AND RELEVANCE

(75) Inventors: Piyush Raizada, Barrington, IL (US); Atulkishen Setlur, Barrington, IL (US); Vadim Berezhanskiy, Barrington, IL (US); Nikhil Jain, Barrington, IL (US); Jeffrey James Whipple, Severn, MD (US)

(73) Assignee: General Electric Company, Schenectady, NY (US)

(21) Appl. No.: 12/979,640

(22) Filed: Dec. 28, 2010

Related U.S. Application Data
(60) Provisional application No. 61/417,200, filed on Nov. 24, 2010.

(51) Int. Cl. G06Q 50/00; G06Q 10/00

(52) U.S. Cl. 705/2

(57) ABSTRACT

An example method includes receiving an update message regarding a first exam record; updating the first exam record based on the message; matching one or more additional exam records to the first exam record based on one or more predefined exam attributes; selecting one of the exam first record and the one or more additional exam records as an eligible exam record; hiding display of the one or more exam records not selected as the eligible exam record; displaying the eligible exam record; receiving an additional update message for the first exam record; evaluating the additional update message to determine applicability of the update message to the matching one or more additional exam records; and selecting one or more of the first exam record and the matching one or more additional exam records as one or more eligible exam records based on evaluating the additional update message.

### RELATED APPLICATIONS

The present application relates to and claims the benefit of priority from U.S. Provisional Patent Application No. 61/417,200, filed on Nov. 24, 2010, which is herein incorporated by reference in its entirety.

### FEDERALLY SPONSORED RESEARCH OR DEVELOPMENT

[Not Applicable]

### MICROFICHE/COPYRIGHT REFERENCE

[Not Applicable]

### FIELD

The presently described technology generally relates to systems and methods to determine performance indicators in a workflow in a healthcare enterprise. More particularly, the presently described technology relates to computing operation metrics for patient and exam workflow.

### BACKGROUND

Most healthcare enterprises and institutions perform data gathering and reporting manually. Many computerized systems house data and statistics that are accumulated but have to be extracted manually and analyzed after the fact. These approaches suffer from “rear-view mirror syndrome”—by the time the data is collected, analyzed, and ready for review, the institutional makeup in terms of resources, patient distribution, and assets has changed. Regulatory pressures on healthcare continue to increase. Similarly, scrutiny over patient care increases.

Pioneering healthcare organizations such as Kaiser Permanente, challenged with improving productivity and care delivery quality, have begun to define Key Performance Indicators (KPI) or metrics to quantify, monitor and benchmark operational performance targets in areas where the organization is seeking transformation. By aligning departmental and facility KPIs to overall health system KPIs, everyone in the organization can work toward the goals established by the organization.

### BRIEF SUMMARY

Certain examples provide systems, apparatus, and methods for automated tracking and determination of healthcare exam relevance and relationship.

Certain examples provide a computer-implemented method for automated determination of healthcare exam relevance and connectivity. The method includes receiving an update message regarding a first exam record; updating the first exam record based on the message; matching one or more additional exam records to the first exam record based on one or more predefined exam attributes; selecting one of the exam first record and the one or more additional exam records as an eligible exam record; hiding display of the one or more exam records not selected as the eligible exam record; and displaying the eligible exam record. The method includes receiving an additional update message for the first exam record; evaluating the additional update message to determine applicability of the update message to the matching one or more additional exam records; and selecting one or more of the first exam record and the matching one or more additional exam records as one or more eligible exam records based on evaluating the additional update message.

Certain examples provide a tangible computer-readable storage medium having a set of instructions stored thereon which, when executed, instruct a processor to implement a method for automated determination of healthcare exam relevance and connectivity. The method includes receiving an update message regarding a first exam record; updating the first exam record based on the message; matching one or more additional exam records to the first exam record based on one or more predefined exam attributes; selecting one of the exam first record and the one or more additional exam records as an eligible exam record; hiding display of the one or more exam records not selected as the eligible exam record; and displaying the eligible exam record. The method includes receiving an additional update message for the first exam record; evaluating the additional update message to determine applicability of the update message to the matching one or more additional exam records; and selecting one or more of the first exam record and the matching one or more additional exam records as one or more eligible exam records based on evaluating the additional update message.

Certain examples provide a healthcare system including a memory comprising one or more executable instructions and data; a processor to execute the one or more executable instructions and to process the data; and a user interface including a dashboard indicating utilization and performance metrics for a healthcare environment. The processor is to receive an update message regarding a first exam record and update the first exam record based on the message. The processor is to match one or more additional exam records to the first exam record based on one or more predefined exam attributes and select one of the exam first record and the one or more additional exam records as an eligible exam record. The processor is to hide display on the user interface of the one or more exam records not selected as the eligible exam record and displaying the eligible exam record via the user interface. The processor is to receive an additional update message for the first exam record, evaluate the additional update message to determine applicability of the update message to the matching one or more additional exam records, and select one or more of the first exam record and the matching one or more additional exam records as one or more eligible exam records based on evaluating the additional update message.

### BRIEF DESCRIPTION OF SEVERAL VIEWS OF THE DRAWINGS

FIG. 1 depicts an example healthcare information enterprise system to measure, output, and improve operational performance metrics.

FIG. 2 illustrates an example real-time analytics dashboard system.

FIG. 3 illustrates an example dashboard interface to facilitate viewing of and interaction with KPI information, alerts, and other data.

FIG. 4 depicts an example detail patient grid providing patient information and worklist data for a clinician, department, and/or institution, etc.

FIG. 5 illustrates an example dashboard user interface providing outpatient wait times for a healthcare facility.

FIG. 6 illustrates an example dashboard user interface providing delay time and other information for pending exams and/or other procedures for a healthcare facility.

FIG. 7 illustrates an example dashboard user interface providing delay time and other information for pending exams and/or other procedures for a healthcare facility.

FIG. 8 depicts an example digitized whiteboard interface providing an imaging scanner level view of scheduled procedures, utilization, delays, etc.

FIG. 9 depicts an example inquiry view interface for viewing exams scheduled, completed, and in progress.

FIG. 10 depicts a flow diagram for an example method for computation and output of operational metrics for patient and exam workflow.

FIG. 11 illustrates a flow diagram for an example method for exam correlation or linking for performance metric analysis and display.

FIG. 12 illustrates a flow diagram for an example method for exam correlation or linking for performance metric analysis and display.

FIGS. 13-18 illustrate flow diagrams for example methods for exam updating and display with and/or without linking.

FIG. 19 is a block diagram of an example processor system that may be used to implement the systems, apparatus and methods described herein.

The foregoing summary, as well as the following detailed description of certain embodiments of the present invention, will be better understood when read in conjunction with the appended drawings. For the purpose of illustrating the invention, certain embodiments are shown in the drawings. It should be understood, however, that the present invention is not limited to the arrangements and instrumentality shown in the attached drawings.

### DETAILED DESCRIPTION OF CERTAIN EXAMPLES

Although the following discloses example methods, systems, articles of manufacture, and apparatus including, among other components, software executed on hardware, it should be noted that such methods and apparatus are merely illustrative and should not be considered as limiting. For example, it is contemplated that any or all of these hardware and software components could be embodied exclusively in hardware, exclusively in software, exclusively in firmware, or in any combination of hardware, software, and/or firmware. Accordingly, while the following describes example methods, systems, articles of manufacture, and apparatus, the examples provided are not the only way to implement such methods, systems, articles of manufacture, and apparatus.

When any of the appended claims are read to cover a purely software and/or firmware implementation, at least one of the elements in an at least one example is hereby expressly defined to include a tangible medium such as a memory, DVD, CD, Blu-ray, etc. storing the software and/or firmware.

Healthcare has recently seen an increase in a number of information systems deployed. Due to departmental differences, growth paths and adoption of systems have not always been aligned. Departments use departmental systems that are specific to their workflows. Increasingly, enterprise systems are being installed to address some cross-department challenges. Much expensive integration work is required to tie these systems together, and, typically, this integration is kept to a minimum to keep down costs and departments instead rely on human intervention to bridge any gaps.

For example, a hospital may have an enterprise scheduling system to schedule exams for all departments within the hospital. This is a benefit to the enterprise and to patients. However, the scheduling system may not be integrated with every departmental system due to a variety of reasons. Since most departments use their departmental information systems to manage orders and workflow, the department staff has to look at the scheduling system application to know what exams are scheduled to be performed and potentially recreate these exams in their departmental system for further processing.

Certain examples help streamline a patient scanning process in radiology by providing transparency to workflow occurring in disparate systems. Current patient scanning workflow in radiology is managed using paper requisitions printed from a radiology information system (RIS) or manually tracked on dry erase whiteboards. Given the disparate systems used to track patient prep, lab results, oral contrast, it is difficult for Technologists to be efficient, as they need to poll the different systems to check status of patient. Further this information is not easily communicated as it is tracked manually. So any other individual would need to look up this information again or check information via a phone call.

The system provides an electronic interface to display information corresponding to any event in the patient scanning and image interpretation workflow. With visibility to completion on workflow steps in different systems, manually track completion of workflow in the system, visual timer to countdown activity or tasks in radiology.

Certain examples provide electronic systems and methods to capture additional elements that result in delays. Certain example systems and methods capture information electronically including: one or more delay reasons for an exam and/or additional attribute(s) that describe an exam (e.g., an exam priority flag).

Workflow definition can vary from institution to institution. Some institutions track nursing preparation time, radiologist in room time, etc. These states (events) can be dynamically added to a decision support system based on a customer's needs, wants, and/or preferences to enable measurement of key performance indicator(s) (KPI) and display of information associated with KPIs.

Certain examples provide a plurality of workflow state definitions. Certain examples provide an ability to store a number of occurrences of each workflow state and to track workflow steps. Certain examples provide an ability to modify a sequence of workflow to be specific to a particular site workflow. Certain examples provide an ability to cross reference patient visit events with exam events.

Current dashboard solutions are typically based on data in a RIS or picture archiving and communication system (PACS). Certain examples provide an ability to aggregate data from a plurality of sources including RIS, PACS, modality, virtual radiography (VR), scheduling, lab, pharmacy systems, etc. A flexible workflow definition enables example systems and methods to be customized to customer workflow configuration with relative ease.

Additionally, rather than attempting to provide integration between disparate systems, certain examples mimic the rationale used by staff (e.g., configurable per the workflow of a healthcare site) to identify exams in two or more disconnected systems that are the same and/or connected in some way. This allows the site to continue to keep the systems separate but adds value by matching and presenting these exams as a single/same exam, thereby reducing a need for a staff to link exams manually in either system.

Certain examples provide a rules based engine that can be configured to match exams it receives from two or more systems based on user selected criteria to evaluate if these different exams are actually the same exam that is to be performed at the facility. Attributes that can be configured include patient demographics (e.g., name, age, sex, other identifier(s), etc.), visit attributes (e.g., account number, etc.), date of examination, procedure to be performed, etc.

Once two or more exams received from different systems are identified as being the same, single exam, one or more exams are deactivated from the set of linked exams such that only one of the exam entries is presented to an end user. Rather than merging the two exams, a system can be configured to display an exam received from the ordering system and de-activate the exam received from a scheduling system.

For example, when a scheduling system at a hospital is not interfaced with an order entry/management system. When a patient calls to schedule an exam, a record is created in the scheduling system which is then forwarded to a decision support system. Upon arrival of the patient at the hospital, an order is created in the order entry system (e.g., a RIS) to manage an exam-related departmental workflow. This information is also received by the decision support system as a separate exam.

Without an ability to identify related exams and determine which of the related exams should be presented, a decision support dashboard would display two exam entries for what is in reality a single exam. With this capability, the decision support system disables the scheduled exam upon receipt of an order for that patient, preventing both exams from appearing on the dashboard as pending exams. Only the ordered exam is retained. Before the ordered exam information is received, the decision support system displays the scheduled exam.

Thus, a staff user is not required to manually intervene to remove exam entries from a scheduling and/or decision support application. Rather, the exam entry does not progress in a workflow as its ordered counterpart. Behavior of linked or related exams can be customized based on a hospital's workflow without requiring code changes, for example.

Certain examples provide systems and methods to determine operational metrics or key performance indicators (KPIs) such as patient wait time. Certain examples facilitate a more accurate calculation of patient wait time and/or other metric/indicator with a multiple number of patient workflow events to accommodate variation of workflow.

Hospital administrators should be able to quantify an amount of time a patient is waiting during a radiology workflow, for example, where the patient is prepared and transferred to obtain radiology examination by scanners such as magnetic resonance (MR) and/or computed tomography (CT) imaging systems. A more accurate quantification of patient wait time helps to improve patient care and optimize or improve radiology and/or other healthcare department/enterprise operation.

Certain examples help provide an understanding of the real-time operational effectiveness of an enterprise and help enable an operator to address deficiencies. Certain examples thus provide an ability to collect, analyze and review operational data from a healthcare enterprise in real time or substantially in real time given inherent processing, storage, and/or transmission delay. The data is provided in a digestible manner adjusted for factors that may artificially affect the value of the operational data (e.g., patient wait time) so that an appropriate responsive action may be taken.

KPIs are used by hospitals and other healthcare enterprises to measure operational performance and evaluate a patient experience. KPIs can help healthcare institutions, clinicians, and staff provide better patient care, improve department and enterprise efficiencies, and reduce the overall cost of delivery. Compiling information into KPIs can be time consuming and involve administrators and/or clinical analysts generating individual reports on disparate information systems and manually aggregating this data into meaningful information.

KPIs represent performance metrics that can be standard for an industry or business but also can include metrics that are specific to an institution or location. These metrics are used and presented to users to measure and demonstrate performance of departments, systems, and/or individuals. KPIs include, but are not limited to, patient wait times (PWT), turn around time (TAT) on a report or dictation, stroke report turn around time (S-RTAT), or overall film usage in a radiology department. For dictation, a time can be a measure of time from completed to dictated, time from dictated to transcribed, and/or time from transcribed to signed, for example.

In certain examples, data is aggregated from disparate information systems within a hospital or department environment. A KPI can be created from the aggregated data and presented to a user on a Web-enabled device or other information portal/interface. In addition, alerts and/or early warnings can be provided based on the data so that personnel can take action before patient experience issues worsen.

For example, KPIs can be highlighted and associated with actions in response to various conditions, such as, but not limited to, long patient wait times, a modality that is underutilized, a report for stroke, a performance metric that is not meeting hospital guidelines, or a referring physician that is continuously requesting films when exams are available electronically through a hospital portal. Performance indicators addressing specific areas of performance can be acted upon in real time (or substantially real time accounting for processing, storage/retrieval, and/or transmission delay), for example.

In certain examples, data is collected and analyzed to be presented in a graphical dashboard including visual indicators representing KPIs, underlying data, and/or associated functions for a user. Information can be provided to help enable a user to become proactive rather than reactive. Additionally, information can be processed to provide more accurate indicators accounting for factors and delays beyond the control of the patient, the clinician, and/or the clinical enterprise. In some examples, “inherent” delays can be highlighted as separate actionable items apart from an associated operational metric, such as patient wait time.

Certain examples provide configurable KPI (e.g., operational metric) computations in a work flow of a healthcare enterprise. The computations allow KPI consumers to select a set of relevant qualifiers to determine a scope of a data countable in the operational metrics. An algorithm supports the KPI computations in complex work flow scenarios including various work flow exceptions and repetitions in an ascending or descending work flow statuses change order (such as, exam or patient visit cancellations, re-scheduling, etc.), as well as in scenarios of multi-day and multi-order patient visits, for example.

Multiple exams during a single patient visit can be linked based on visit identifier, date, and/or modality, for example. The patient is not counted multiple times for wait time calculation purposes. Additionally, all associated exams are not marked as dictated when an event associated with dictation of one of the exams is received.

Once the above computations are completed, visits and exams are grouped according to one or more time threshold(s) as specified by one or more users in a hospital or other monitored healthcare enterprise. For example, an emergency department in a hospital wants to divide the patient wait times during visits into 0-15 minute, 15-30 minute, and over 30 minute wait time groups.

Once data can be grouped in terms of absolute numbers or percentages, it can be presented to a user. The data can be presented in the form of various graphical charts such as traffic lights, bar charts, and/or other graphical and/or alphanumeric indicators based on threshold(s), etc.

Thus, certain examples help facilitate operational data-driven decision-making and process improvements. To help improve operational productivity, tools are provided to measure and display a real-time (or substantially real-time) view of day-to-day operations. In order to better manage an organization's long-term strategy, administrators are provided with simpler-to-use data analysis tools to identify areas for improvement and monitor the impact of change. For example, imaging departments are facing challenges around reimbursement. Certain examples provide tool to help improve departmental operations and streamline reimbursement documentation, support, and processing.

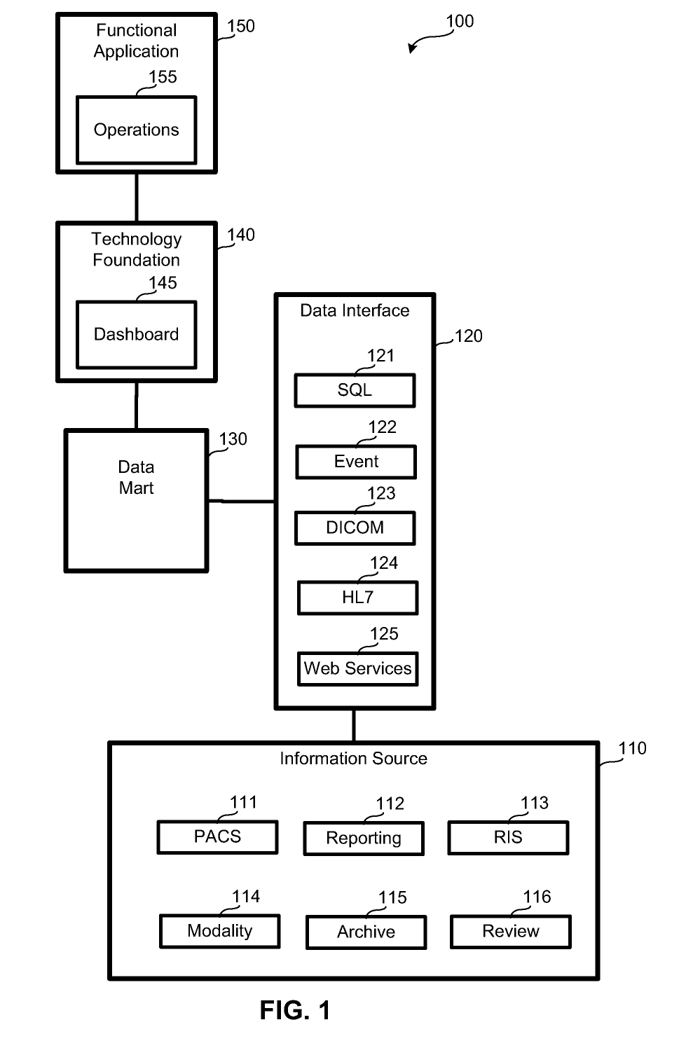

FIG. 1 depicts an example healthcare information enterprise system 100 to measure, output, and improve operational performance metrics. The system 100 includes a plurality of information sources, a dashboard, and operational functional applications. More specifically, the example system 100 shown in FIG. 1 includes a plurality of information sources 110 including, for example, a picture archiving and communication system (PACS) 111, a precision reporting subsystem 112, a radiology information system (RIS) 113 (including data management, scheduling, etc.), a modality 114, an archive 115, a modality 116, and a quality review subsystem 116 (e.g., PeerVue™)

The plurality of information sources 110 provide data to a data interface 120. The data interface 120 can include a plurality of data interfaces for communicating, formatting, and/or otherwise providing data from the information sources 110 to a data mart 130. For example, the data interface 120 can include one or more of an SQL data interface 121, an event-based data interface 122, a DICOM data interface 123, an HL7 data interface 124, and a web services data interface 125.

The data mart 130 receives and stores data from the information source(s) 110 via the interface 120. The data can be stored in a relational database and/or according to another organization, for example. The data mart 130 provides data to a technology foundation 140 including a dashboard 145. The technology foundation 140 can interact with one or more functional applications 150 based on data from the data mart 130 and analytics from the dashboard 145, for example. Functional applications can include operations applications 155, for example.

As will be discussed further below, the dashboard 145 includes a central workflow view and information regarding KPIs and associated measurements and alerts, for example. The operations applications 155 include information and actions related to equipment utilization, wait time, report read time, number of cases read, etc.

KPIs reflect the strategic objectives of the organization. Examples in Radiology include but are not limited to reduction in patient wait times, improving exam throughput, reducing dictation and report turn-around times, and increasing equipment utilization rate. KPIs are used to assess the present state of the organization, department or the individual and to provide actionable information with a clear course of action. They assist a healthcare organization to measure progress towards the goals and objectives established for success. Departmental managers and other front-line staff, however, find it difficult to pro-actively manage to these KPIs in real-time. This is at least partly because the data to build KPIs resides in disparate information sources and should be correlated to compute KPI performance.

A KPI can accommodate, but is not limited to, the following workflow scenarios:

1. Patient wait times until an exam is started.

2. Turn-around times between any hospital workflow states.

3. Add or remove multiple exam/patient states from KPI computations. For example, some hospitals wish to add multiple lab states in a patient workflow, and KPI computations can account for these states in the calculations.

4. Canceled visits and exams should automatically be excluded from computations.

5. Multiple exams in single patient visit during single day should be distinguished from single patient wait time versus single patient same exam during multiple days.

6. Wait time deductions should be applied where drugs are administered and drugs take time to come into affect.

7. Off business hours should be excluded from turn around and/or wait times of different events.

8. Exam should be allowed to roll back into any previous state and should be excluded or included in KPI calculations accordingly.

9. A user should have options to configure KPI according to hospital needs/wants/preferences, and KPI should perform calculations according to user configurations.

10. Multiple exams should be linked to single exams if the exams are from a single visit, same modality, same patient, and same day, for example.

Using KPI computation(s) and associated support, a hospital and/or other healthcare administrator can obtain more accurate information of patient wait time and/or turn-around time between different workflow states in order to optimize or improve operation to provide better patient care.

Even if a patient workflow involves an alternate workflow, the application can obtain multiple workflow events to process a more accurate patient wait time. Calculation of patient wait time or turn-around time between different workflow states can be configured and adjusted for different workflow and procedures.

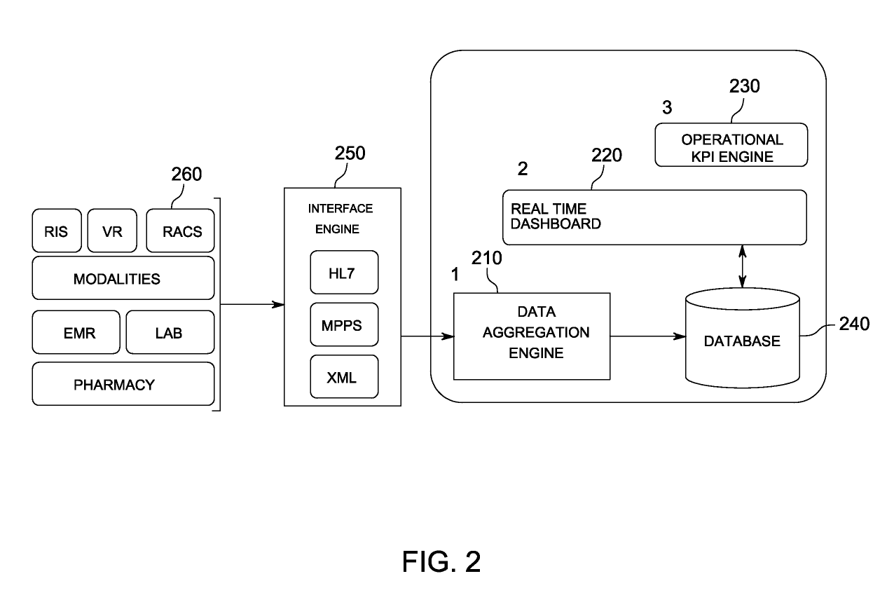

FIG. 2 illustrates an example real-time analytics dashboard system 200. The real-time analytics dashboard system 200 is designed to provide radiology and/or other healthcare departments with transparency to operational performance around workflow spanning from schedule (order) to report distribution.

The dashboard system 200 includes a data aggregation engine 210 that correlates events from disparate sources 260 via an interface engine 250. The system 200 also includes a real-time dashboard 220, such as a real-time dashboard web application accessible via a browser across a healthcare enterprise. The system 200 includes an operational KPI engine 230 to pro-actively manage imaging and/or other healthcare operations. Aggregated data can be stored in a database 240 for use by the real-time dashboard 220, for example.

The real-time dashboard system 200 is powered by the data aggregation engine 210, which correlates in real-time (or substantially in real time accounting for system delays) workflow events from PACS, RIS, and other information sources, so users can view status of patient within and outside of radiology and/or other healthcare department(s).

The data aggregation engine 210 has pre-built exam and patient events, and supports an ability to add custom events to map to site workflow. The engine 210 provides a user interface in the form of an inquiry view, for example, to query for audit event(s). The inquiry view supports queries using the following criteria within a specified time range: patient, exam, staff, event type(s), etc. The inquiry view can be used to look up audit information on an exam and visit events within a certain time range (e.g., six weeks). The inquiry view can be used to check a current workflow status of an exam. The inquiry view can be used to verify staff patient interaction audit compliance information by cross-referencing patient and staff information.

The interface engine 250 (e.g., a CCG interface engine) is used to interface with a variety of information sources 260 (e.g., RIS, PACS, VR, modalities, electronic medical record (EMR), lab, pharmacy, etc.) and the data aggregation engine 210. The interface engine 250 can interface based on HL7, DICOM, XML, MPPS, and/or other message/data format, for example.

The real-time dashboard 220 supports a variety of capabilities (e.g., in a web-based format). The dashboard 220 can organize KPI by facility and allow a user to drill-down from an enterprise to an individual facility (e.g., a hospital). The dashboard 220 can display multiple KPI simultaneously (or substantially simultaneously), for example. The dashboard 220 provides an automated “slide show” to display a sequence of open KPI. The dashboard 220 can be used to save open KPI, generate report(s), export data to a spreadsheet, etc.

The operational KPI engine 230 provides an ability to display visual alerts indicating bottleneck(s) and pending task(s). The KPI engine 230 computes process metrics using data from disparate sources (e.g., RIS, modality, PACS, VR, etc.). The KPI engine 230 can accommodate and process multiple occurrences of an event and access detail data under an aggregate KPI metric, for example. The engine 230 can specify a user-defined filter and group by options. The engine 230 can accept customized KPI thresholds, time depth, etc., and can be used to build custom KPI to reflect a site workflow, for example.

KPI generated can include a turnaround time KPI, which calculates a time taken from one or more initial workflow states to complete one or more final states, for example. The KPI can be presented as an average value on a gauge or display counts grouped into turnaround time categories on a stacked bar chart, for example.

A wait time KPI calculates an elapsed time from one or more initial workflow states to a current time until a set of final workflow states have not been completed, for example. This KPI is visualized in a traffic light displaying counts of exams grouped by time thresholds, for example.

A comparison or count KPI computes counts of exams in one state versus another state for a given time period. Alternatively, counts of exams in a single state can be computed (e.g., a number of cancelled exams). This KPI is visualized in the form of a bar chart, for example.

The dashboard system 200 can provide graphical reports to visualize patterns and quickly identify short-term trends, for example. Reports are defined by, for example, process turnaround times, asset utilization, throughput, volume/mix, and/or delay reasons, etc.

The dashboard system 200 can also provide exception outlier score cards, such as a tabular list grouped by facility for a number of exams exceeding turnaround time threshold(s).

The dashboard system 200 can provide a unified list of pending emergency department (ED), outpatient, and/or inpatient exams in a particular modality (e.g., department) with an ability to: 1) display status of workflow events from different systems, 2) indicate pending multi-modality exams for a patient, 3) track time for a certain activity related to an exam via countdown timer, and/or 4) electronically record Delay Reasons, a Timestamp for the occurrence of a workflow event, for example.

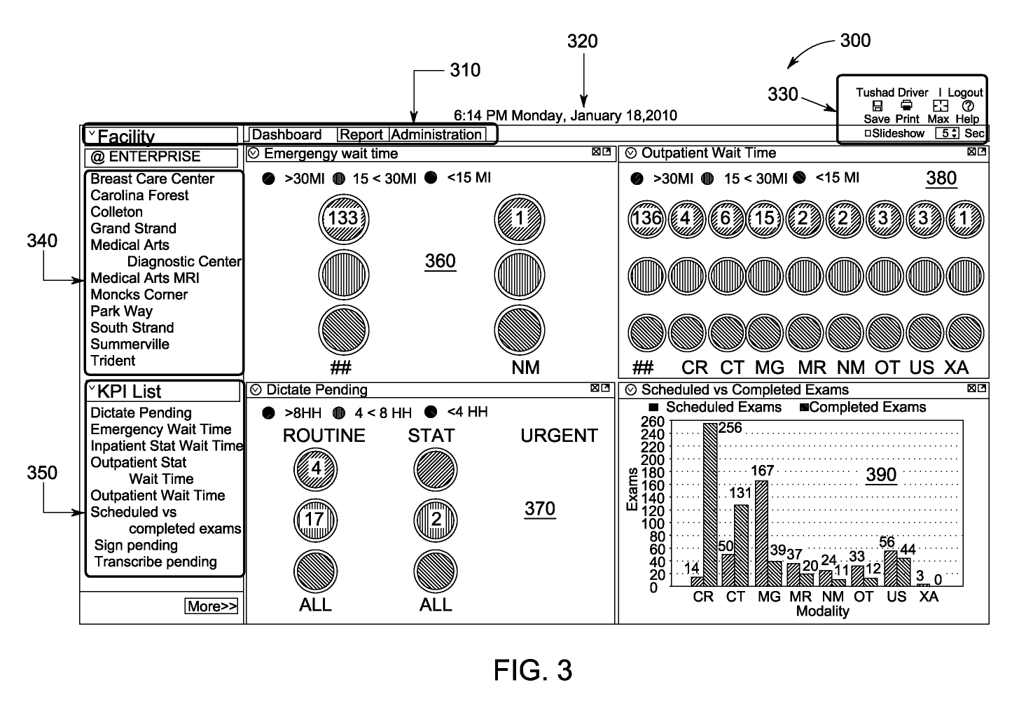

FIG. 3 illustrates an example dashboard interface 300 to facilitate viewing of and interaction with KPI information, alerts, and other data. The dashboard 300 provides a real-time (or at least substantially real-time) view of radiology and/or other department and/or enterprise operations tailored to administrator, technologist, wait areas, and/or other criteria, etc. The dashboard 300 helps facilitate pro-active management via visual and off-line alert and helps to streamline communication. The dashboard can be Web-based and/or accessible via other software application on a user's computer, for example.

The dashboard 300 can help provide seamless (or relatively seamless) access to workflow status, for example. The dashboard 300 can receive data from a robust correlation engine that aggregates workflow events from a variety of sources including a modality, PACS, RIS, virtual radiography (VR), labs, pharmacy/pharmaceutical, scheduling, computerized physician order entry (CPOE). The dashboard 300 can provide facility level data segregation (e.g., views, multi-RIS, etc.). In certain examples, the dashboard 300 presents collected information and allows a user to view and drill down to further levels of detail regarding the information. The dashboard 300 can be configurable based on institution, department, user, etc.

For example, at an enterprise level, users can monitor financial data from billing and cost tracking systems, average census information, number of admissions and discharges, and length of stay. At a departmental level, users can monitor patient wait times, average number of exams performed, types of exams performed, dictation and report turn-around times, and employee utilization. At an individual level, performance of staff, equipment and support systems, as well as overall patient, physician and employee satisfaction, can be monitored. In certain examples, the dashboard 300 can be a part of an Internet web site or system to facilitate collaboration and exchange of KPIs and related data among an online community.

Additionally, the dashboard 300 can help facilitate ongoing performance improvement for a healthcare facility. For example, a custom workflow definition can be developed to more accurately represent cross-departmental workflow and customize facility-specific process metrics. A monthly outlier report can help capture reason(s) for delay.

The example dashboard 300 includes a tab control 310 to facilitate user navigation between modules in the dashboard (e.g., dashboard, report, administration, etc.). The dashboard 300 also includes a header 320 to provide identification information such as time, date, user, role, etc. The dashboard 300 includes one or more convenience controls 330 to allow a user to quickly access and execute certain functionality such as save KPI, print KPI, expand KPI, help, slide show, etc.

The dashboard 300 includes a tree control 340 to facilitate navigation through healthcare facilities in a particular region or market. For example, the navigation control 340 can include a plurality of facilities in a region or common ownership structure and allow a user to select one or more of the regions to display KPIs and/or other information associated with the selected facility(ies).

The dashboard 300 also includes a KPI selection control 350. One or more KPIs 360, 370, 380, 390 are displayed in more detail via the dashboard 300 based on one or more of default settings, user preferences, and/or selections via the KPI selection control 350. For example, a user can select one or more KPIs for which information has been collected and processed including but not limited to dictation pending, emergency wait time, in-patient STAT wait time, out-patient wait time, scheduled versus completed exams, signature pending, and/or transcription pending, etc.

As shown, for example, in FIG. 3, an emergency wait time KPI 360 is depicted using a visual “traffic light” representation of KPI data and associated alerts. Visual cues provide an indication of how many patients have been waiting less than fifteen minutes (green), between fifteen and thirty minutes (yellow), and more than thirty minutes (red) (e.g., one shown in the example dashboard 300) for a computed tomography (CT) or computed radiography (CR) exam. Thus, the circles in the KPI box 360 are lights that show the status of that indicator based upon one or more pre-determined parameters (e.g., green for good, yellow or amber for caution or possible problems, and red for an alert condition or existence of a significant problem). In certain examples, by selecting one of the circles, additional information regarding the associated data and metric/parameter used to analyze it can be displayed to the user. Other visual and/or alphanumeric alert indicators can be used instead of or in addition to the traffic light indicators shown in FIG. 3.

As shown, for example, in FIG. 3, a dictation pending KPI 370 is also depicted using a visual traffic light representation of KPI data and associated alerts. Visual cues provide an indication of how many exams have been sitting in a queue for less than four hours (green), between four and eight hours (yellow), and more than eight hours (red) to be reviewed and have results dictated. In the example of FIG. 3, four routine exams have been waiting for more than eight hours; seventeen routine and two stat exams have been waiting between four and eight hours; and no exams have been waiting in the queue for less than four hours.

As shown, for example, in FIG. 3, an outpatient wait time KPI 380 is depicted using a visual traffic light representation of KPI data and associated alerts. Visual cues provide an indication of how many outpatients have waiting to be seen for less than fifteen minutes (green), between fifteen and thirty minutes (yellow), and more than thirty minutes (red). In the example of FIG. 3, several patients have been waiting for more than thirty minutes for a variety of services, such as CR, CT, mammography (MG), MR, nuclear medicine (NM), other (OT), ultrasound (US), and/or X-ray angiography (XA).

As shown, for example, in FIG. 3, a scheduled versus completed exams KPI 390 is represented using a bar graph and associated numbers. The bars of the bar graph are colored to indicate scheduled exams versus completed exams. The bar provides a visual indication of a number of exams in relation to a y axis of a number of exams and an x axis of modality (e.g., CR, CT, MG, MR, NM, OT, US, XA, etc.). An alphanumeric indicator can also be displayed to provide an exact number of exams associated with the data point. Thus, a breakdown of pending versus completed exams can be provided by modality.

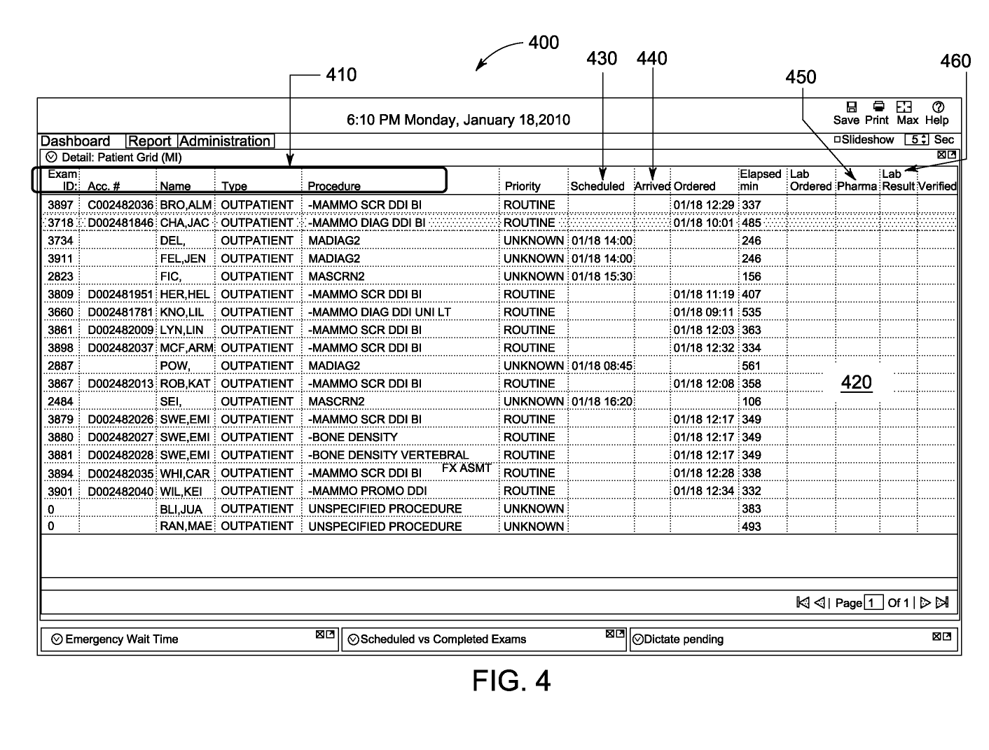

FIG. 4 depicts an example detail patient grid 400 providing patient information and worklist data for a clinician, department, and/or institution, etc. The patient grid 400 can be access via a tab control 410 and/or other option in the dashboard 400, for example. The patient grid 400 includes patient information 410 including exam identifier (ID), account number, name, type (e.g., outpatient, inpatient, emergency, etc.), procedure, priority, etc. The patient information 410 can include patient name and/or be anonymized depending upon user access and privacy rights. The patient information 410 can combine or separate inpatient, outpatient, and/or department (e.g., emergency department (ED)) patients in the view 400.

The patient grid 400 includes a data grid 420 associated with the patient information 410. The data grid 420 provides information and details timestamps indicating workflow state completion, for example. In certain examples, items in the data grid 420 can be selected (e.g., mouse/cursor click, mouseover, etc.) to display further information and/or associated functionality.

The grid 400 also displays a scheduled time 430 for a patient in the patient list. The schedule time 430 can include a link to access a scheduling interface, for example. The example grid 400 shows patient arrival, discharge, and/or transfer (ADT) information 440 as well. Other information such as procedure order date/time, lab order date/time, pharma information 450 (e.g., a contrast pull), lab results 460, verification information, etc., can be provided in the data grid 420.

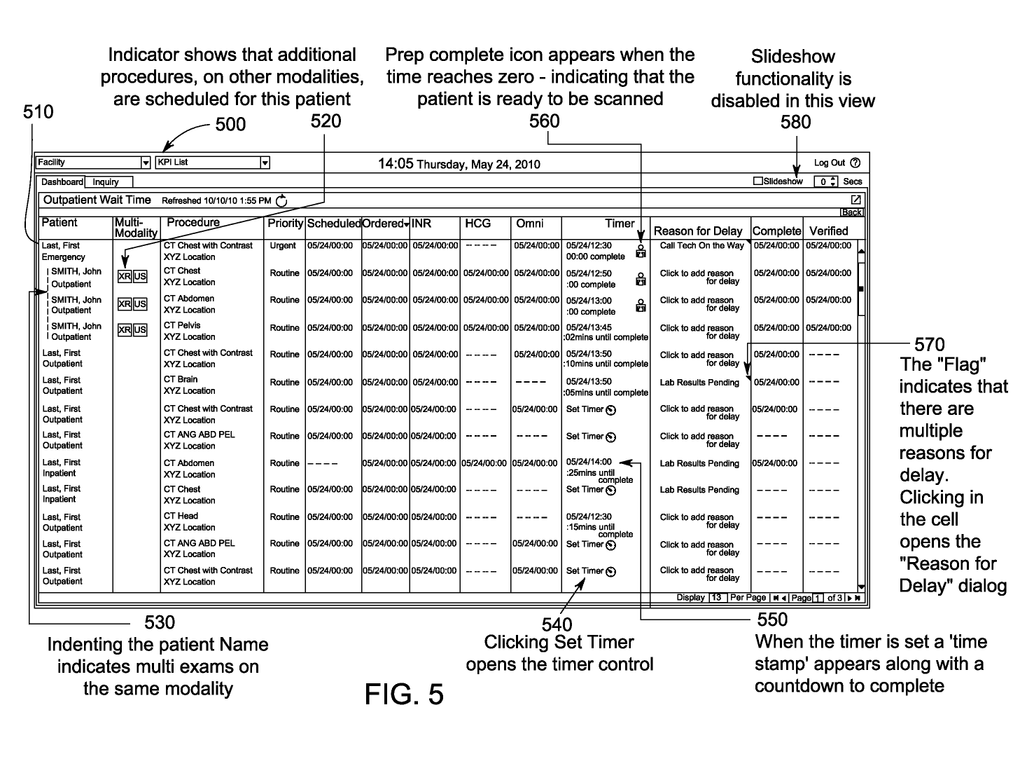

FIG. 5 illustrates an example dashboard user interface 500 providing wait time and other information for pending exams and/or other procedures for a healthcare facility. The dashboard 500 includes a listing of one or more patients 510 with information about those patients at the facility. For example, patient name and/or other identification is provided along with modality(ies), procedure and location, priority, scheduled time, ordered time, timer, reason for delay, completion time, verification time, etc.

A multi-modality indicator 520 shows that multiple procedures on multiple modalities (e.g., X-ray, ultrasound, CT, MR, etc.) are scheduled for a patient. Multiple listings for a patient 530 indicate multiple exams. As depicted in the example of FIG. 5, indenting the patient name 530 indicates multiple exams on the same modality (e.g., a chest CT, an abdominal CT, and a pelvic CT at the same location).

The example interface 500 includes a timer 540 indicating a time until a scheduled procedure is completed. Using the interface 500, a user can open a timer 540 to set the timer for a procedure preparation using a timer control. For example, a time to prepare scanning equipment can be accounted for using the timer. A time to allow contrast ingestion/injection by the patient to take effect can be tracked using the timer, for example. A time for anesthesia to take effect can be tracked using the timer, for example. When a timer is set, a time stamp 550 appears along with a countdown to preparation completion, as illustrated in the example of FIG. 5. As shown in the example of FIG. 5, a preparation complete icon 560 appears with the timer 540 reaches zero, indicating that the patient is ready for the procedure (e.g., ready to be scanned).

As shown in the example interface 500, a flag 570 indicates that there are multiple reasons for delay for a patient and/or an associated procedure. Selecting the flag opens an interface dialog or window providing additional detail regarding the reasons for delay.

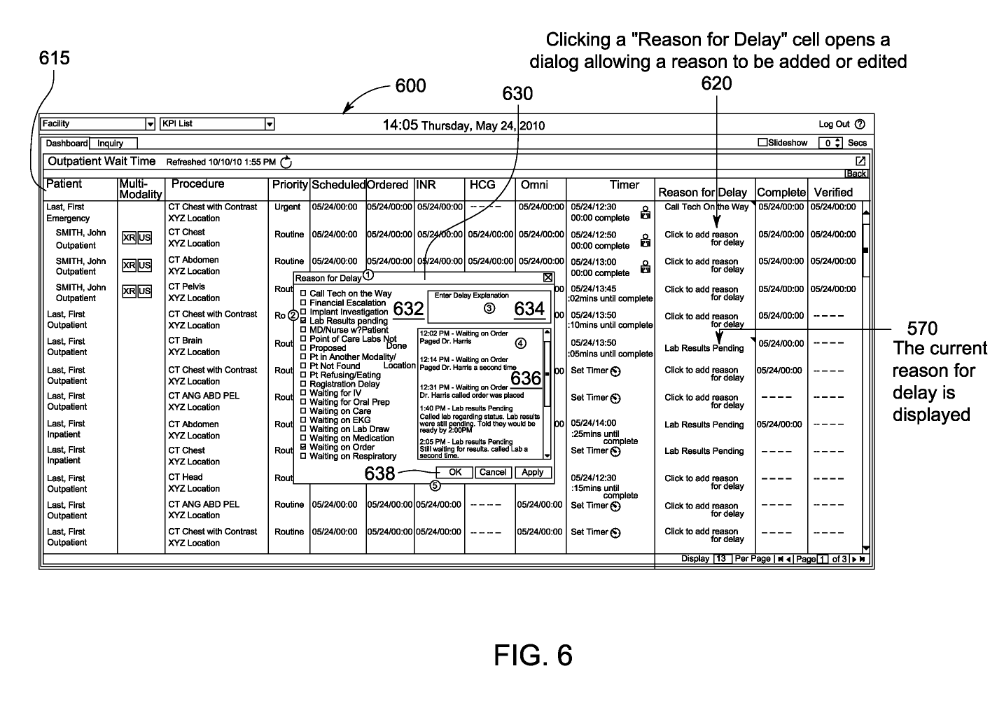

FIG. 6 illustrates an example dashboard user interface 600 providing delay time and other information for pending exams and/or other procedures for a healthcare facility. The interface includes a current reason for delay 610 listed for each patient/procedure entry in the interface table 615. Selecting a reason for delay entry 620 opens an interface dialog or window 630 allowing one or more reasons for delay to be added and/or edited, for example.

The reason for delay dialog box 630 includes a selectable list 632 of preset reasons for delay that is selectable by a user, for example. A user can select one or more reasons from the list 632, for example. Additionally, a user can manually enter an explanation for delay 634. This text field 634 allows a user to replace and/or supplement delay information associated with a selected reason from the list 632, for example. The dialog 630 also includes a delay event log 634. When a reason for delay is checked and applied, for example, the reason and a time stamp are entered into the log 634, along with any explanation provided by the user. One or more dialog buttons 636 can be used to apply multiple reasons and/or explanations to the log 634 and interface 610, close the dialog 630 with changes, cancel without making changes, etc.

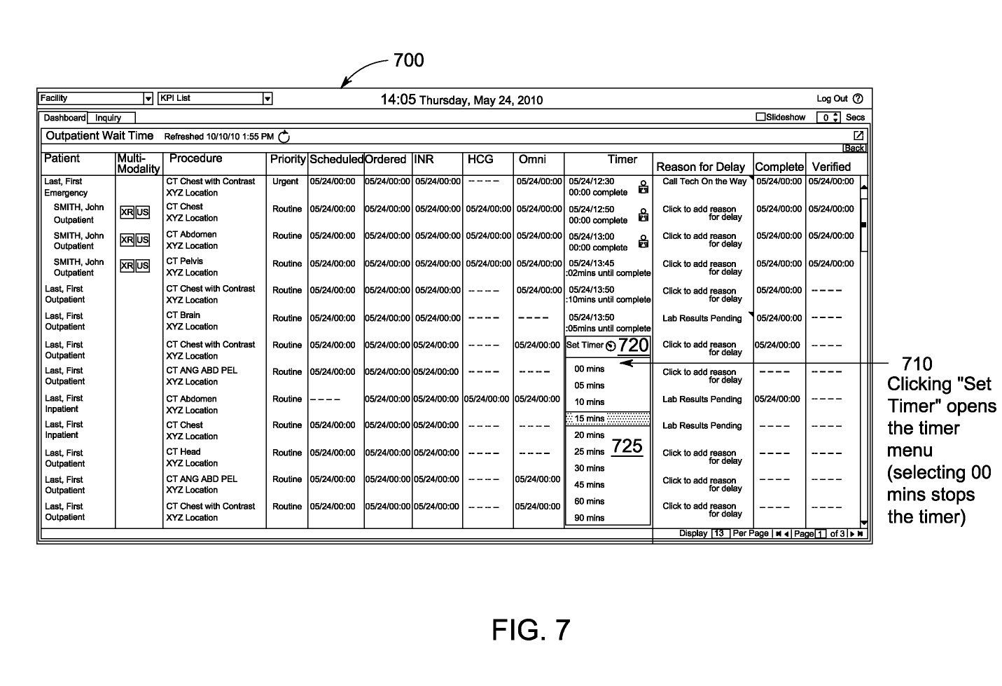

FIG. 7 illustrates an example dashboard user interface 700 providing delay time and other information for pending exams and/or other procedures for a healthcare facility. As shown in the example interface 700, selecting a timer entry 710 opens a set timer menu. The set timer menu 720 includes a plurality of time values 725 for selection by a user. Selecting zero minutes, for example, stops the timer.

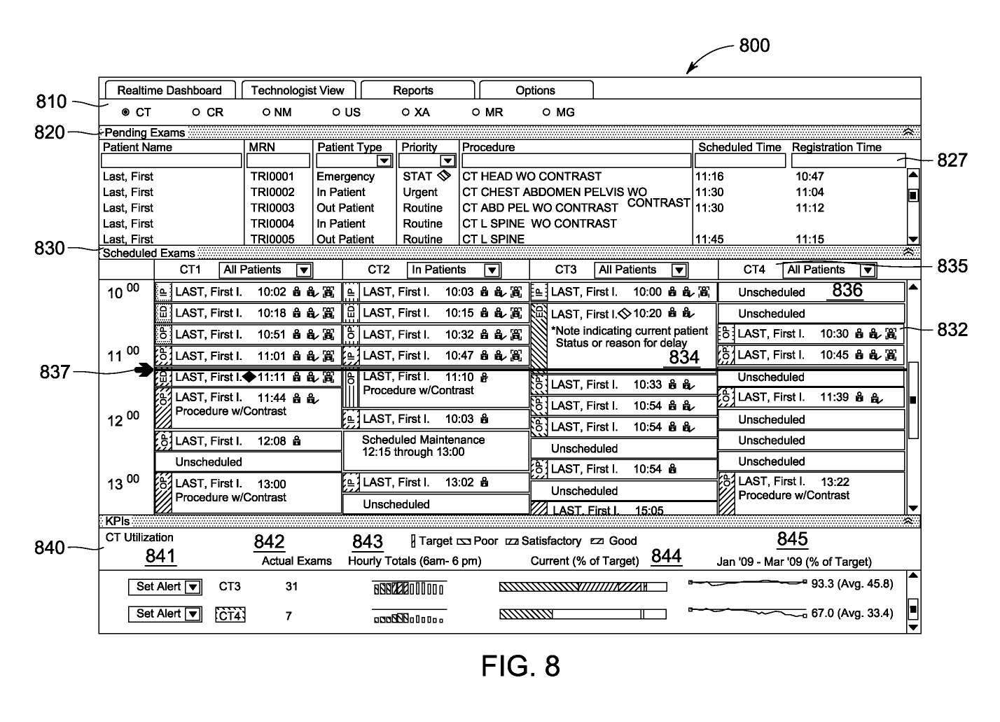

FIG. 8 depicts an example digitized whiteboard interface 800 providing an imaging scanner level view of scheduled procedures, utilization, delays, etc. The example interface 800 provides a selectable listing of exams by modality 810. Exams are separated in the example of FIG. 8 into pending exams 820 and scheduled exams 830. One or more KPIs 840 can be provided based on the exam information.

The listing of pending exams 820 includes a listing by patient 825 that can be automatically retrieved from one or more information systems/scanners and/or manually entered 827 by a user, for example. A patient type, priority, procedure, and difference between registration time and scheduled time can be noted, for example.

The listing of scheduled exams 830 separates exams based on available equipment 835, for example. A current time 837 can be graphically indicated (e.g., using a line) in the schedule 830, for example. For each patient on a given equipment 835, a graphical presentation of pending state(s) 832 can be provided. In certain examples, one or more icons can be used to represent a current state/status. Icons can include patient arrived, nursing preparation started, nursing preparation completed, patient ready, patient scan in progress, etc. Additionally, a visual indication of delay(s) 834 can be represented as they occur. A graphical representation of open slot(s) 836 can also be provided, as shown in the example of FIG. 8.

One or more KPIs 840 can be configured and/or provided via the example interface 800. Using the example interface 800, a machine utilization (e.g., CT utilization) KPI can be set by setting an alert 841 for a particular machine. An actual number of exams 842 associated with a machine can be provided, for example. An hourly total of exams/machine usage 843 can be represented. A current utilization 844 (e.g., a percentage of a target utilization) is shown in the example interface 800 of FIG. 8. Additionally, a usage over time 845 (e.g., a percentage of target utilization) can be provided.

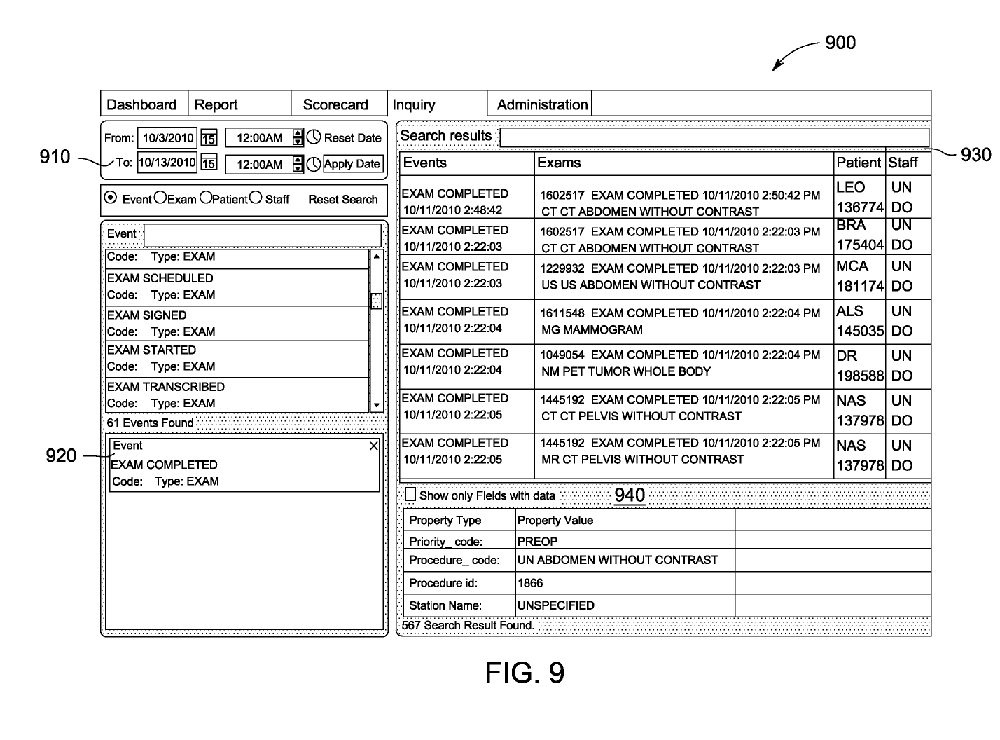

FIG. 9 depicts an example inquiry view interface 900 for viewing exams scheduled, completed, and in progress. The inquiry view 900 can be used to search for one or more of scheduled exams, completed exams, exams in progress, etc. The inquiry view 900 can be useful for audit compliance checks (e.g., to reference staff and/or patient workflow(s), etc.). Additionally, the inquiry view 900 can be used to look up multi-system workflow events (e.g., current exam status, exam and/or patient workflow event(s), etc.).

The example inquiry view interface 900 includes a search control 910, applied search criteria 920, search results 930, and detail 940 regarding a selected search result. One or more search criteria 920 can be specified by a user, for example. Results can be organized according to one or more criterion such as event, exam, patient, staff, etc. Applied search criteria 930 are displayed to the user, for example. Search results 930 are provided for user review and selection. Search results 930 can include information such as reference number, current exam status, last event time, procedure information, patient name, patient identification number, staff identification, etc. A result can be selected to display further detail 940 regarding that result, for example.

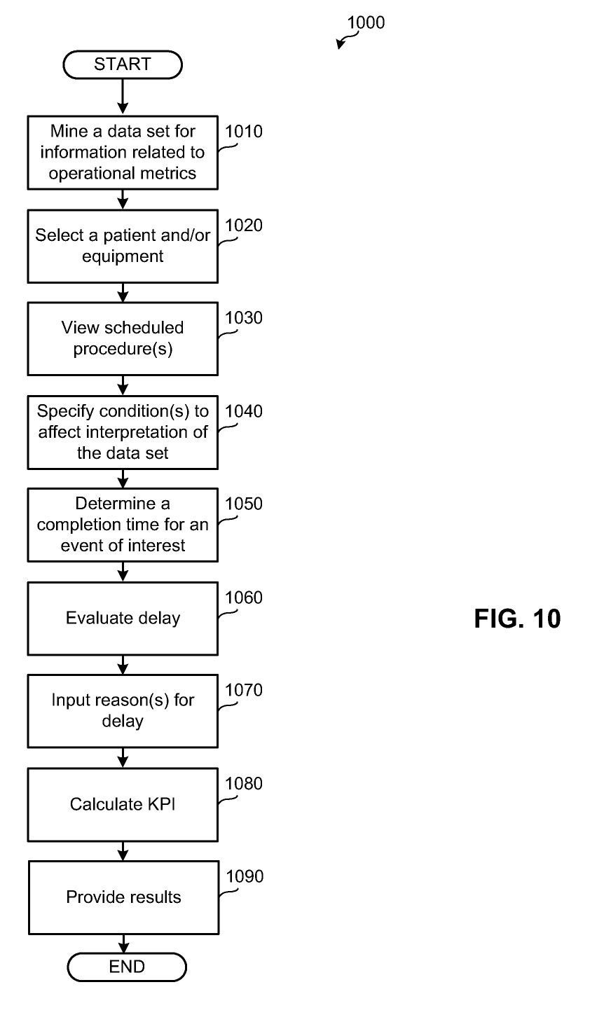

FIG. 10 depicts an example flow diagram representative of process(es) that can be implemented using, for example, computer readable instructions that can be used to facilitate collection of data, calculation of KPIs, and presentation for review of the KPIs. The example process(es) of FIG. 10 can be performed using a processor, a controller and/or any other suitable processing device. For example, the example processes of FIG. 10 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example process(es) of FIG. 10 can be implemented using coded instructions (e.g., computer readable instructions) stored on a non-transitory computer readable medium such as a flash memory, a read-only memory (ROM), a random-access memory (RAM), a CD, a DVD, a Blu-ray, a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example process(es) of FIG. 10 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example process(es) of FIG. 10 can be implemented manually or as any combination(s) of any of the foregoing techniques, for example, any combination of firmware, software, discrete logic and/or hardware. Further, although the example process(es) of FIG. 10 are described with reference to the flow diagram of FIG. 10, other methods of implementing the processes of FIG. 10 may be employed. For example, the order of execution of the blocks can be changed, and/or some of the blocks described may be changed, eliminated, sub-divided, or combined. Additionally, any or all of the example process(es) of FIG. 10 can be performed sequentially and/or in parallel by, for example, separate processing threads, processors, devices, discrete logic, circuits, etc.

FIG. 10 depicts a flow diagram for an example method 1000 for computation and output of operational metrics for patient and exam workflow. At block 1010, an available data set is mined for information relevant to one or more operational metrics. For example, an operational data set obtained from multiple information sources, such as image modality and medical record archive data sources, are mined at both an exam and a patient visit level within a specified time range based on initial and final states of patient visit and exam workflow. This data set includes date and time stamps for events of interest in a hospital workflow along with exam and patient attributes specified by standards/protocols, such as HL7 and/or DICOM standards.

At block 1020, one or more patient(s) and/or equipment of interest are selected for evaluation and review. For example, one or more patients in one or more hospital departments and one or more pieces of imaging equipment (e.g., CT scanners) are selected for review and KPI generation. At block 1030, scheduled procedures are displayed for review.

At block 1040, a user can specify one or more conditions to affect interpretation of the data in the data set. For example, the user can specify whether any or all states relevant to a workflow of interest have or have not been reached. For example, the user also has an ability to pass relevant filter(s) that are specific to a hospital workflow. A resulting data set is built dynamically based on the user conditions.

At block 1050, a completion time for an event of interest is determined. At block 1060, a delay associated with the event of interest is evaluated. At block 1070, one or more reasons for delay can be provided. For example, equipment setup time, patient preparation time, conflicted usage time, etc., can be provided as one or more reasons for a delay.

At block 1080, one more KPIs can be calculated based on the available information. At block 1090, results are provided (e.g., displayed, stored, routed to another system/application, etc.) to a user.

Thus, certain examples provide systems and methods to assist in providing situational awareness to steps and delays related to completion of patient scanning workflow. Certain examples provide a current status of patient in a scanning process, electronically recorded delay reasons, and a KPI computation engine that aggregates and provides data for display via a user interface. Information can be presented in a tabular list and/or a calendar view, for example. Situational awareness can include patient preparation (e.g., oral contrast administered/dispense time), lab results and/or order result time, nursing preparation start/complete time, exam order time, exam schedule time, patient arrival time, etc.

Given the dynamic nature of workflow in healthcare institutions, time stamps can be tracked for custom states. Certain examples provide an extensible way to track workflow events, with minimal effort. An example operational metrics engine also tracks the current state of an exam, for example. Activities shown on a dashboard (whiteboard) result in tracking time stamp(s), communicating information, and/or automatically changing state based on one or more rules, for example. Certain examples allow custom addition of states and associated color and/or icon presentation to match customer workflow, for example.

Most organizations lack electronic data for delays in workflow. In certain examples, a real-time dashboard allows tracking of multiple delay reasons for a given exam via reason codes. Reason codes are defined in a hierarchical structure with a generic set that applies across all modalities, extended by modality specific reason codes, for example. This allows presenting relevant delay codes for a given modality

Certain examples provide an ability to support multiple occurrences of a single workflow step (e.g., how many times a user entered an application/workflow and did something, did nothing, etc.). Certain examples provide an ability to select a minimum, a maximum, and/or a count of multiple times that a single workflow step has occurred. Certain examples provide a customizable workflow definition and/or an ability to correlate multiple modality exams. Certain examples provide an ability to track a current state of exam across multiple systems.

Certain examples provide an extensible workflow definition wherein a generic event can be defined which represents any state. An example engine dynamically adapts to needs of a customer without planning in advance for each possible workflow of the user. For example, if a user's workflow is defined today to include A, B, C, and D, the definition can be dynamically expanded to include E, F, and G and be tracked, measured, and accommodated for performance without creating rows and columns in a workflow state database for each workflow eventuality in advance.

This information can be stored in a row of a workflow state table, for example. Data can be transposed dynamically from a dashboard based on one or more rules, for example. For example, a KPI rules engine can take a time stamp, such as an ordered time stamp, a scheduled time stamp, an arrived time stamp, a completed time stamp, a verified time stamp, etc., and each category of time stamp that as an event type associated with a number of occurrences. A user can select a minimum or maximum of an event, track multiple occurrences of an event, count a number of events by patient and/or exam, track patient visit level event(s), etc.

Frequently, multiple tests are ordered for a single patient, and these tests are viewed on exam lists filtered for a given modality without any indicator of the other modality exams. This leads to “waste” in patient transport as, quite often, the patient is returned to the original location rather than being handed off from one modality to another. A real-time dashboard provides a way to correlate multiple modality exams at a patient level and display one or more corresponding indicator(s), for example. For example, multiple modalities can be cross-referenced to show that a patient has an x-ray, CT, and ultrasound all scheduled to happen in one day.

In certain example, not only are time stamps captured and metrics presented, but accompanying delay reasons, etc., are captured and accounted for as well. In addition to system-generated timestamps, a user can interact and add a delay reason in conjunction with the timestamp, for example.

In certain examples, when computing KPIs, a modality filter is excluded upon data selection. Data is grouped by visit and/or by patient identifier, selecting aggregation criteria to correlate multi-modality exams, for example. Data can be dynamically transposed, for example. The example analysis returns only exams for the filtered modality with multi modality indicators.

Certain examples provide systems and methods to identify, prioritize, and/or synchronize related exams and/or other records. In certain examples, messages can be received for the same domain object (e.g., an exam) from different sources. Based on customer created rules, the objects (e.g., exams) are matched such that it is confidently determine that two or more exam records belonging to different systems actually represent the same exam, for example.

Based on the information included in the exam records, one of the exam records is selected as the most eligible/applicable record, for example. By selecting a record, a corresponding source system is selected whose record is to be used, for example. In some examples, multiple records can be selected and used. Other, non-selected matching records are hidden from display. These hidden exams are linked to the displayed exam implicitly based on rules. In certain examples, there is no explicit linking via references, etc.

Matching exams in a set progress in lock-step through the workflow, for example. When a status update is received for one exam in the set, all exams are updated to the same status together. In certain examples, this behavior applies only to status updates. In certain examples, due to updates to an individual exam record from its source system (other than a status update), if an updated exam no longer matches with the linked set of exams, it is automatically unlinked from the other exams and moves (progresses/regresses) in the workflow independently. In certain examples, due to updates to an individual exam record from its source system, a hidden exam may become displayed and/or a displayed exam may become hidden based on events and/or rules in the workflow.

For example, exams received from the same system are automatically linked based on set criteria. Thus, an automated behavior can be created for exams when an ordering system cannot link the exams during ordering.

In certain examples, two or more exams for the same study are linked at a modality by a technologist when performing an exam. From then on, the exams move in lock-step through the imaging workflow (not the reporting workflow). This is done by adding accession numbers (e.g., unique identifiers) for the linked exams in the single study's DICOM header. Systems capable of reading DICOM images can infer that the exams are linked from this header information, for example. However, these exams appear as separate exams in a pre-imaging workflow, such as patient wait and preparation for exams, and in post imaging workflow, such as reporting (e.g., where systems are non-DICOM compatible).

For example, using a dashboard, a CT chest, abdomen and pelvis display as three different exams. The three exams are performed together in a single scan. Since each exam is displayed independently, there is possibility of dual work (e.g., ordering additional labs if the labs are tied to the exams). Certain examples link two or more exams from the same ordering system that are normally linked and for different procedures using set of rules created by a customer such that these exams show up and progress through pre- and post-imaging workflow as linked exams. By linked exams, two or more exam records are counted as one exam since they are to be acquired/performed in the same scanning session, for example.

Exam correlation or “linking” helps reduce a potential for multiple scans when a single scan would have sufficed (e.g., images for all linked exams could have been captured in a single scan). Exam correlation/relationship helps reduce staff workload and errors in scheduling (e.g., scheduling what is a single scan across multiple days because of more than one order). Exam correlation helps reduces potential for additional radiation, additional lab work, etc. Doctors are increasingly ordering exams covering more parts of body in a single scan, especially in trauma cases, for example. Such correlation or relational linking provides a truer picture of a department workload by differentiating between scan and exam. Scan is a workflow item (not an exam), for example.

Thus, certain examples use rule-based matching of two or more exams (e.g., from the same or different ordering systems, which can be part of a rule itself) to determine whether the exams should be linked together to display as a single exam on a performance dashboard. Without such rule-based matching, a user would see two or three different exams waiting to be done for what in reality is only a single scan, for example.

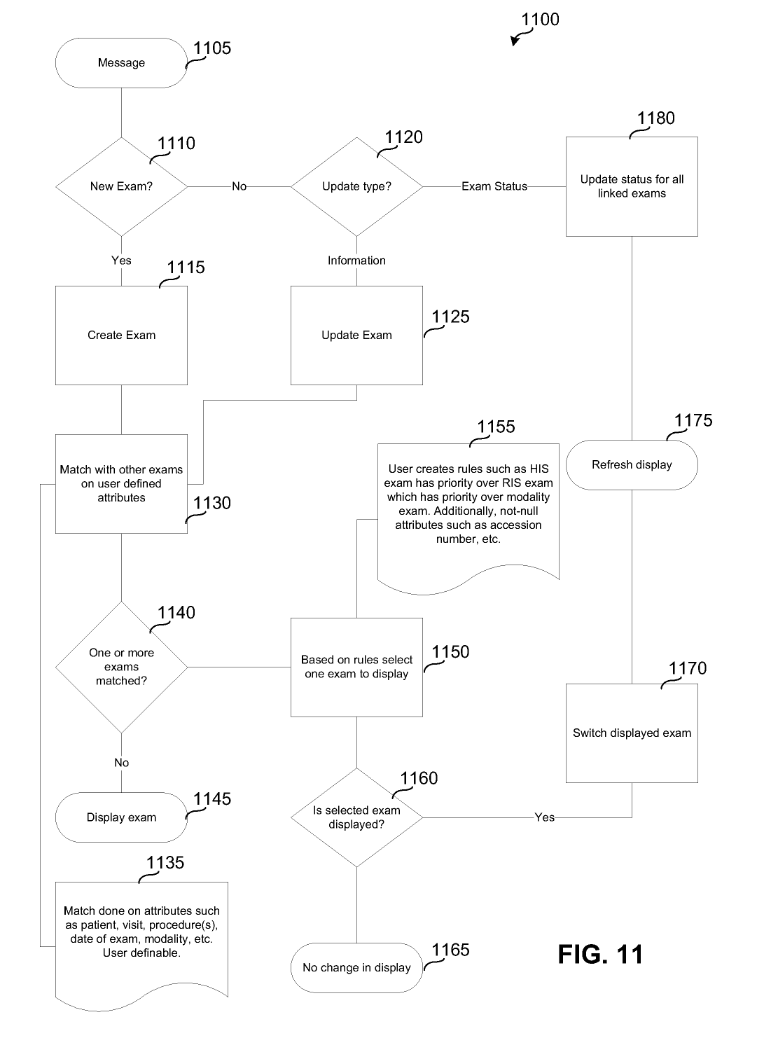

FIGS. 11-18 depict example flow diagrams representative of processes that can be implemented using, for example, computer readable instructions that can be used to facilitate collection of data, calculation of KPIs, and presentation for review. The example processes of FIGS. 11-18 can be performed using a processor, a controller and/or any other suitable processing device. For example, the example processes of FIGS. 11-18 can be implemented using coded instructions (e.g., computer readable instructions) stored on a tangible computer readable medium such as a flash memory, a read-only memory (ROM), and/or a random-access memory (RAM). As used herein, the term tangible computer readable medium is expressly defined to include any type of computer readable storage and to exclude propagating signals. Additionally or alternatively, the example processes of FIGS. 11-18 can be implemented using coded instructions (e.g., computer readable instructions) stored on a non-transitory computer readable medium such as a flash memory, a read-only memory (ROM), a random-access memory (RAM), a CD, a DVD, a Blu-ray, a cache, or any other storage media in which information is stored for any duration (e.g., for extended time periods, permanently, brief instances, for temporarily buffering, and/or for caching of the information). As used herein, the term non-transitory computer readable medium is expressly defined to include any type of computer readable medium and to exclude propagating signals.

Alternatively, some or all of the example processes of FIGS. 11-18 can be implemented using any combination(s) of application specific integrated circuit(s) (ASIC(s)), programmable logic device(s) (PLD(s)), field programmable logic device(s) (FPLD(s)), discrete logic, hardware, firmware, etc. Also, some or all of the example processes of FIGS. 11-18 can be implemented manually or as any combination(s) of any of the foregoing techniques, for example, any combination of firmware, software, discrete logic and/or hardware. Further, although the example processes of FIGS. 11-18 are described with reference to the flow diagram of FIGS. 11-18, other methods of implementing the processes of FIGS. 11-18 may be employed. For example, the order of execution of the blocks can be changed, and/or some of the blocks described may be changed, eliminated, sub-divided, or combined. Additionally, any or all of the example processes of FIGS. 11-18 can be performed sequentially and/or in parallel by, for example, separate processing threads, processors, devices, discrete logic, circuits, etc.

FIG. 11 illustrates a flow diagram for an example method 1100 for exam correlation or linking for performance metric analysis and display.

At block 1105, a message is received for a domain object (e.g., an exam). At block 1110, it is determined whether the message is associated with a new exam. If the exam is a new exam, then, at block 1115, the new exam object is created.

If the exam is not a new exam, then, at block 1120, the message is evaluated to determine what type of update is represented by the message. If the update is an information update, then, at block 1125, the exam record is updated based on the information in the message. If the update is an exam status update, then, at block 1180, a status is updated for all exams linked to the exam in question.

At block 1130, the exam is matched with other exam(s) based on one or more user-defined attributes. For example, as shown at block 1135, matching is done based on attributes such as patient, visit, procedure(s), date of exam, modality, etc. Attributes can be user definable, for example.

At block 1140, it is determined whether one or more exams match the exam in question. If not, then, at block 1145, the exam is displayed. If yes, then, at block 1150, one or more relevant exams are selected for display from among the group of matching exams based on one or more rules. For example, as shown at block, 1155, a user can create rule(s) such as a HIS has priority over a RIS exam, which has priority over a modality exam record, etc. Additionally, non-null attributes such as accession number, etc., can be used to determine a relevant exam.

At block 1160, the selected exam(s) are evaluated to determine whether they are already displayed. If not, then, at block 1165, the display is updated to show the exam record(s). If the selected exam record(s) are already being displayed, then, at block 1170, a displayed exam is switched and/or supplemented by the selected relevant exam(s).

At block 1175, the display is refreshed based on the updated exam information. At block 1180, status information is updated for all linked exams.

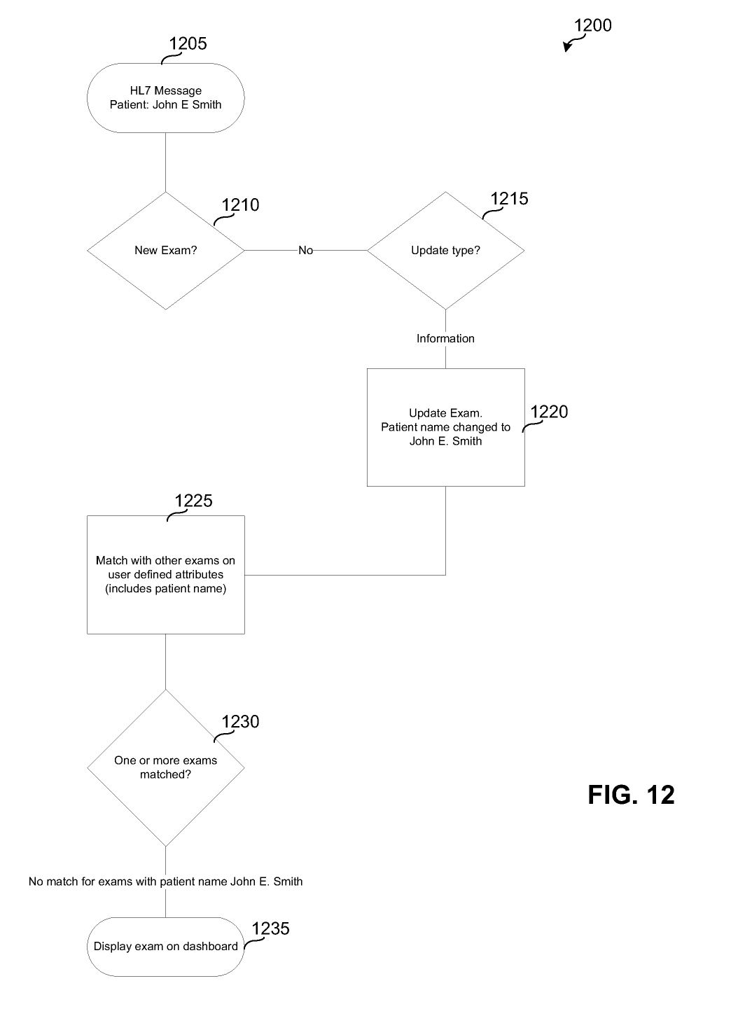

FIG. 12 depicts a flow diagram for an example method 1200 for automatically unlinking an exam after information update. In the example of FIG. 12, three exams for patient John Smith are linked. An exam from the HIS is displayed based on customer defined display rules and other two exams are hidden. An HL7 message is received for one of the exams with the patient name changed to John E. Smith.

At block 1205, an HL7 message is received for a patient John E. Smith. At block 1210, the message is evaluated to determine if it is associated with a new exam. At block 1215, since the message is associated with an existing exam, a type of update associated with the message is determined. The message in the example is determined to be an informational update message.

At block 1220, the exam record is updated to change the name of the patient from John Smith to John E. Smith. At block 1225, the exam is matched with other exams based on user defined attributes (e.g., patient name John E. Smith). At block 1230, a number of matching exams is examined. In the example, no exams were found with a patient name of John E. Smith. At block 1235, the exam is automatically unlinked from the other two exams and displayed independently on the dashboard. The example dashboard shows two exams, one exam for patient John Smith and one exam for John E. Smith.

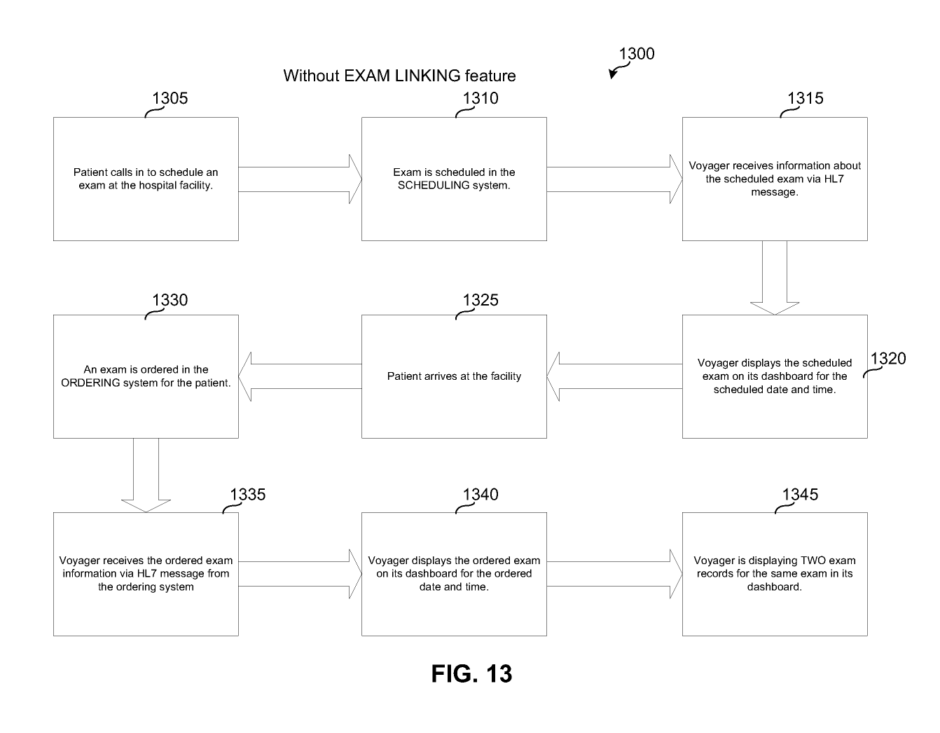

FIGS. 13-18 illustrate flow diagrams for example methods 1300, 1400, 1500, 1600, 1700, and 1800 for exam updating and display with and/or without linking.

The example method 1300 of FIG. 13 provides an example of exam display on a dashboard without linking. At block 1305, a patient calls a hospital to schedule an exam at the hospital facility. At block 1310, the exam is scheduled in a scheduling system. At block 1315, a dashboard/performance monitoring system receives information about the scheduled exam via a message (e.g., an HL7 message).

At block 1320, the performance monitoring system displays the scheduled exam on a dashboard for the scheduled date and time. At block 1325, the patient arrives at the facility. At block 1330, an exam is ordered in an ordering system for the patient.

At block 1335, the performance monitoring system receives the ordered exam information via message (e.g., HL7 message) from the ordering system. At block 1340, the performance monitoring system displays the ordered exam on the dashboard for the ordered date and time.

At block 1345, the performance monitoring system is displaying two exam records for the same exam in its dashboard.

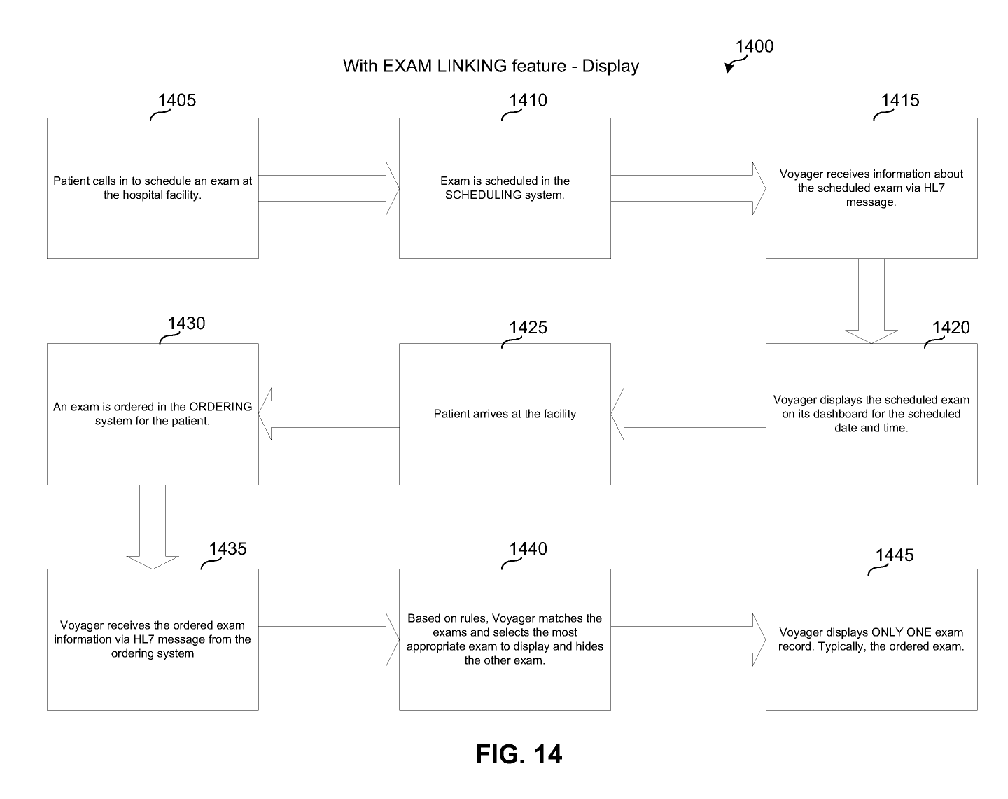

In contrast, the example method 1400 of FIG. 14 provides an example of exam display on a dashboard with linking. At block 1405, a patient calls a hospital to schedule an exam at the hospital facility. At block 1410, the exam is scheduled in a scheduling system. At block 1415, a dashboard/performance monitoring system receives information about the scheduled exam via a message (e.g., an HL7 message).

At block 1420, the performance monitoring system displays the scheduled exam on a dashboard for the scheduled date and time. At block 1425, the patient arrives at the facility. At block 1430, an exam is ordered in an ordering system for the patient.

At block 1435, the performance monitoring system receives the ordered exam information via message (e.g., HL7 message) from the ordering system. At block 1440, based on one or more rules, the performance monitoring system matches the exams and selects a most appropriate exam to display and hides the other exam.

At block 1445, the performance monitoring system is displaying only one exam record in its dashboard. For example, the ordered exam can be selected for display via the dashboard.

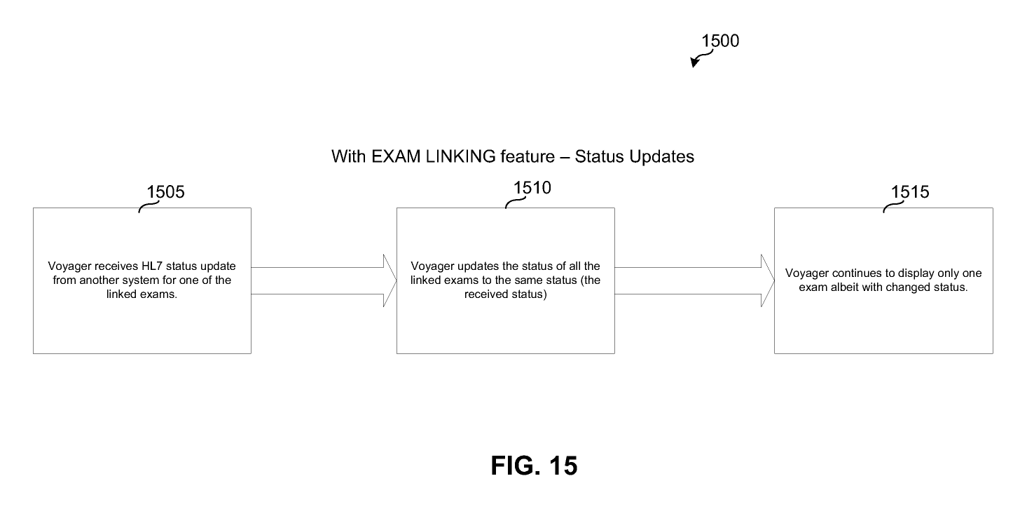

The example method 1500 of FIG. 15 provides an example of exam status update for linked exams. At block 1505, a performance monitoring system receives a status update (e.g., an HL7 message with a status update) from another system for one of a number of linked exams. At block 1510, the performance monitoring system updates the status of all of the linked exams to the same status (the received status). At block 1515, the performance monitoring system continues to display only one exam, albeit with changed status.

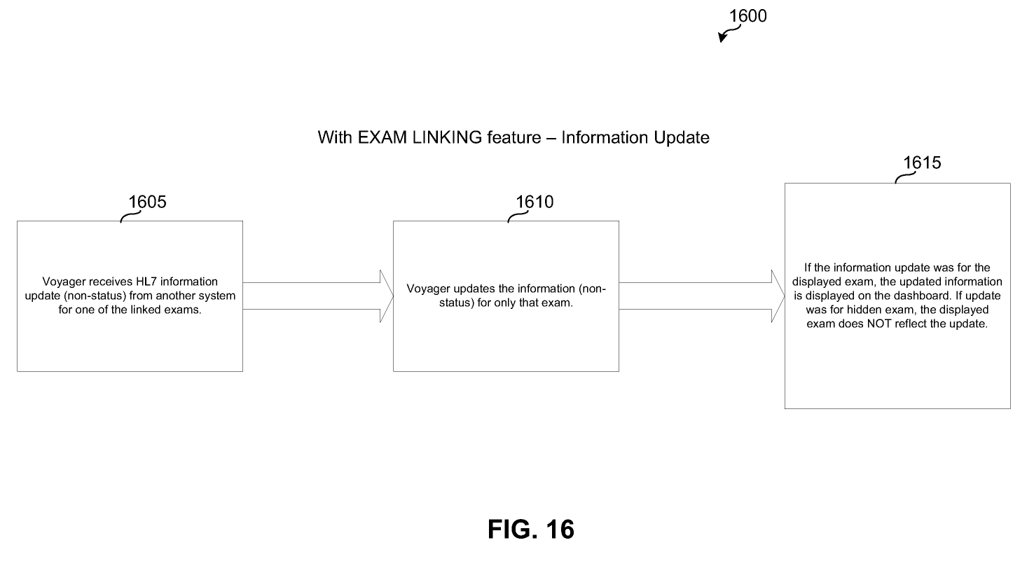

The example method 1600 of FIG. 16 provides an example of exam information update for linked exams. At block 1605, a performance monitoring system receives an information update (e.g., an HL7 message with a non-status information update) from another system for one of a number of linked exams. At block 1610, the performance monitoring system updates the information (non-status) for only that exam. At block 1615, if the information update was for the displayed exam, the updated information is displayed on the dashboard. If the update was for a hidden exam, the displayed exam does not reflect the update.

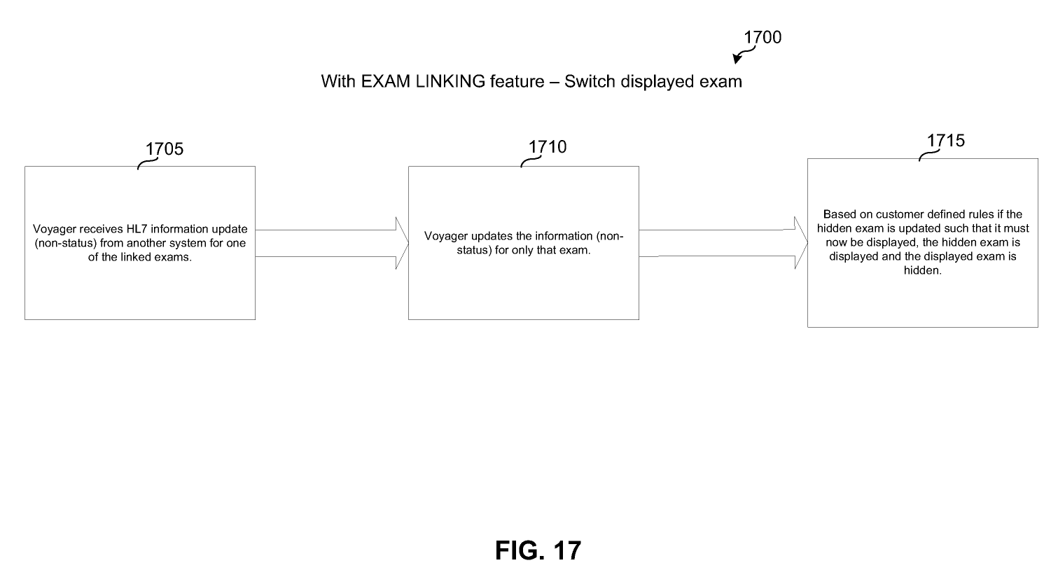

The example method 1700 of FIG. 17 provides an example of exam information update for linked exams. At block 1705, a performance monitoring system receives an information update (e.g., an HL7 message with a non-status information update) from another system for one of a number of linked exams. At block 1710, the performance monitoring system updates the information (non-status) for only that exam. At block 1715, based on customer defined rules, if the hidden exam is updated such that it must now be displayed, the hidden exam is displayed, and the displayed exam is hidden. If the information update was for the displayed exam, the updated information is displayed for the displayed exam on the dashboard.

For example, this situation can occur in cases where the owner of the accession number is the HIS and the RIS created an emergency order without getting that information from the HIS. At this point, there may be three exam records—one from the HIS, one from the RIS and one from the modality itself. All of these exams are linked by the performance monitoring solutions, and determination of which one to display is based on site configured rules. A priority (in the rules) can be HIS then RIS and then modality's unspecified exam, for example.

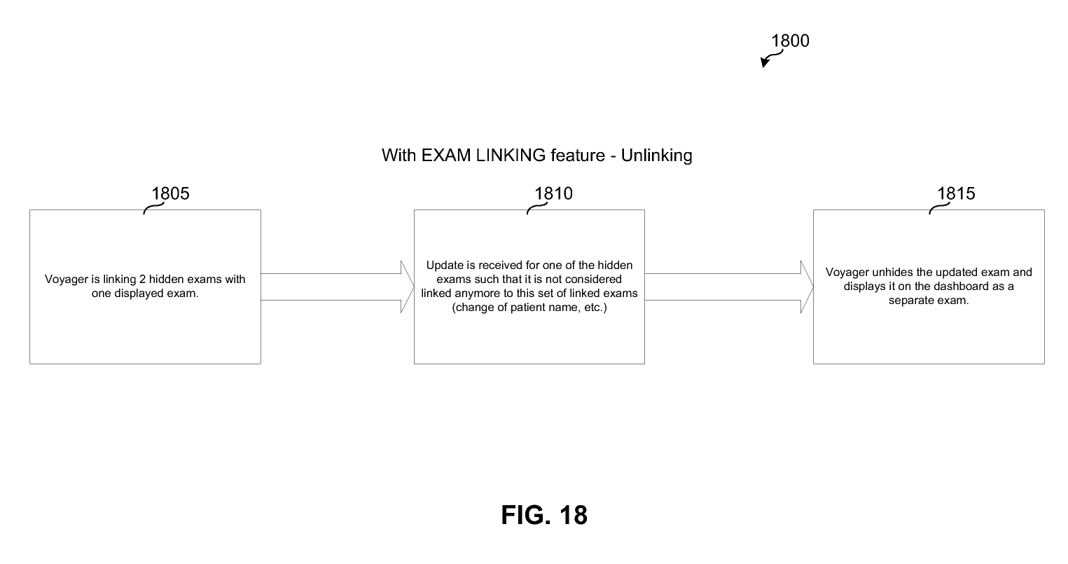

The example method 1800 of FIG. 18 provides an example of exam unlinking following update. At block 1805, a performance monitoring system is linking two hidden exams with one displayed exam. At block 1810, an update is received for one of the hidden exams such that it is not considered linked anymore to this set of linked exams (e.g., change of patient name, etc.). At block 1815, the system unhides the updated exam and displays the exam on the dashboard as a separate exam.

Thus, in certain examples, multiple systems manage different aspects of the patient workflow at a hospital. The systems are not typically integrated, leading to pieces of information about the patient workflow scattered across the multiple systems. As a result, information about the patient workflow is not updated in all the systems in a timely and accurate fashion, which can lead to costly user errors depending on in which system they view their information. Multiple instances of the same patient workflow potentially lead to inaccurate estimation of pending work at a facility. For example, at a site with un-integrated scheduling and RIS systems, the scheduled exam and ordered exam may be for the same patient visit but can lead to an estimation of two different exams on that day. Multiple exams that can be performed with a single scan may be erroneously scheduled on different days causing potential for excessive radiation and reduced income on the scan for the site.

Thus, rather than requiring that end users view information on the patient workflow in each of multiple systems to get a complete picture or manually intervene to remove one of the multiple exam entries, example systems and methods described herein apply rules to available information regarding related exams to help ensure that one exam entry does not progress in the workflow as its ordered counterpart. Related exams can be linked and/or unlinked depending upon the circumstance and/or changes to exam-related data, for example.

Rather than actually merging related and/or duplicate records, certain examples help eliminate a need for a merge in which a staff member at the hospital searches for exams from different systems and then matches and merges them. Without this merge, the system would display all the exam records from all the systems giving an impression of a higher workload. This manual merge operation can take a human staff member three minutes or more per exam, for example. At a midsized hospital with one hundred or more such exams in a day, it requires a full resource to manage this merge of exams. Conversely, by providing a rules-based ability to relate, link and unlink exams, a need for this additional resource is removed. Certain examples also help to remove or reduce merge mistakes due to user error.

By relatably linking exams with an ability to unlink those exams based on changing circumstances/information, both exams continue to exist, owned by their respective creator systems. Both exams continue to receive updates from their creating systems, respectively. Neither exam is updated with information from the other exam. This offers an advantage over standard merge when it is identified that these exams should not in fact be linked with each other. In certain examples, an update to a hidden exam can change the exam such that it no longer matches with a displayed exam. This automatically causes the hidden exam to be displayed again on the dashboard with its updated information. The same cannot be said when the exams are actually merged.

In certain examples, by allowing this capability to be user configurable, linking/relational behavior can be customized based on a hospital's workflow without requiring code changes. This leads to shorter implementation time and an ability to change system behavior as a workflow evolves over time.

Thus, in certain examples, exams in question are neither merged (either manually or automatically) nor linked explicitly. Linking of exams and a decision regarding display of a correct/most applicable exam(s) is made on each refresh of data in a datastore to be displayed on a screen to a user, for example.

Messages can be received for the same domain object (e.g., an exam) from different sources. Based on customer created rules, the exams are matched such that a user can confidently determine that two or more exam records from different systems actually represent the same exam. Matching is done on customer identified exam attributes such as patient name, age, sex, date of birth, etc., and government identifiers such as social security number, etc. In certain examples, parameters such as optionality, priority, weight, etc., can be assigned to attributes.

Based on the information contained in the exam records, one of the exam records is selected as the most eligible record, and, thus, the corresponding source system whose record will be used is selected. Display of other matching exam records is hidden, but the hidden exams are linked to the displayed exam implicitly based on rules. The exams progress through a patient workflow, and, when a status update is received for one exam in the set, all exams are updated to the same status together. However, individual exam record updates provided by the exam's source system are not propagated to other “linked” exams. As a result of an update to an exam record, it may no longer match with the linked set of exams. If so, the non-matching record is automatically unlinked from other exams, displayed, and tracked independently with respect to the patient workflow, for example. Thus, due to updates to an individual exam record from its source system, a hidden exam can be displayed and/or a displayed exam can be hidden.

Customers define rules for when an exam becomes most eligible for display within a set of linked exams. For example, this can be done by assigning priority to the source system. For example, exam records from a hospital information system (HIS) are displayed if available, with corresponding records from other system(s) being hidden. In the absence of a record from the HIS, the exam record from a RIS takes priority, after which the exam from a modality takes priority, etc., for example.

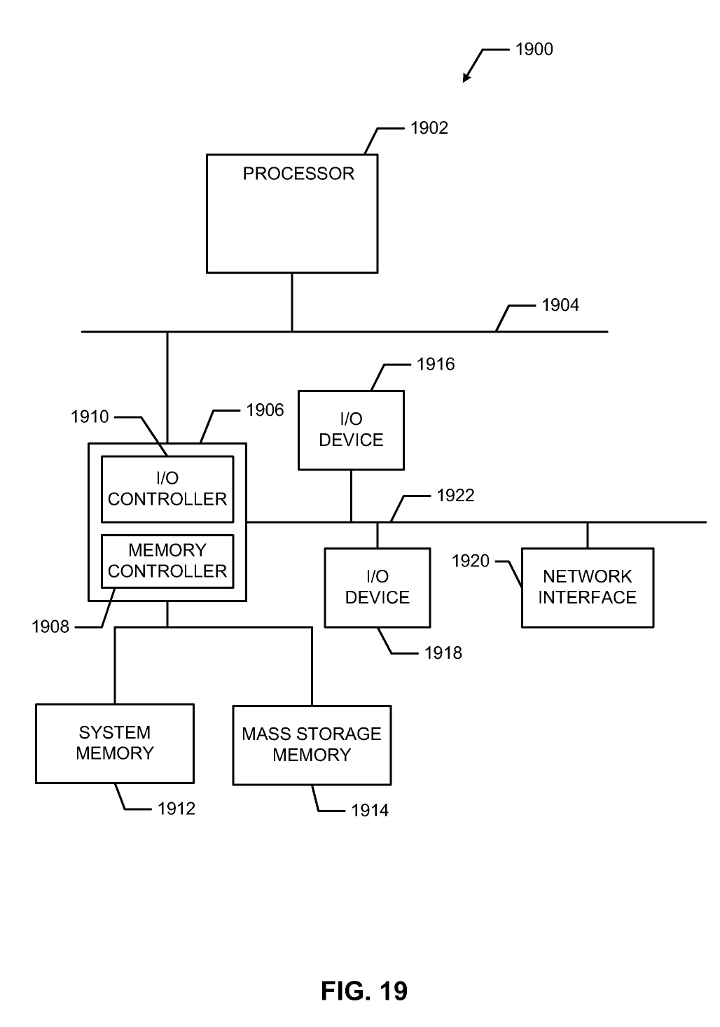

FIG. 19 is a block diagram of an example processor system 1910 that may be used to implement the systems, apparatus and methods described herein. As shown in FIG. 19, the processor system 1910 includes a processor 1912 that is coupled to an interconnection bus 1914. The processor 1912 may be any suitable processor, processing unit or microprocessor. Although not shown in FIG. 19, the system 1910 may be a multi-processor system and, thus, may include one or more additional processors that are identical or similar to the processor 1912 and that are communicatively coupled to the interconnection bus 1914.

The processor 1912 of FIG. 19 is coupled to a chipset 1918, which includes a memory controller 1920 and an input/output (I/O) controller 1922. As is well known, a chipset typically provides I/O and memory management functions as well as a plurality of general purpose and/or special purpose registers, timers, etc. that are accessible or used by one or more processors coupled to the chipset 1918. The memory controller 1920 performs functions that enable the processor 1912 (or processors if there are multiple processors) to access a system memory 1924 and a mass storage memory 1925.

The system memory 1924 may include any desired type of volatile and/or non-volatile memory such as, for example, static random access memory (SRAM), dynamic random access memory (DRAM), flash memory, read-only memory (ROM), etc. The mass storage memory 1925 may include any desired type of mass storage device including hard disk drives, optical drives, tape storage devices, etc.

The I/O controller 1922 performs functions that enable the processor 1912 to communicate with peripheral input/output (I/O) devices 1926 and 1928 and a network interface 1930 via an I/O bus 1932. The I/O devices 1926 and 1928 may be any desired type of I/O device such as, for example, a keyboard, a video display or monitor, a mouse, etc. The network interface 1930 may be, for example, an Ethernet device, an asynchronous transfer mode (ATM) device, an 802.11 device, a DSL modem, a cable modem, a cellular modem, etc. that enables the processor system 1910 to communicate with another processor system.

While the memory controller 1920 and the I/O controller 1922 are depicted in FIG. 19 as separate blocks within the chipset 1918, the functions performed by these blocks may be integrated within a single semiconductor circuit or may be implemented using two or more separate integrated circuits.

Certain embodiments contemplate methods, systems and computer program products on any machine-readable media to implement functionality described above. Certain embodiments may be implemented using an existing computer processor, or by a special purpose computer processor incorporated for this or another purpose or by a hardwired and/or firmware system, for example.

One or more of the components of the systems and/or steps of the methods described above may be implemented alone or in combination in hardware, firmware, and/or as a set of instructions in software, for example. Certain embodiments may be provided as a set of instructions residing on a computer-readable medium, such as a memory, hard disk, DVD, or CD, for execution on a general purpose computer or other processing device. Certain embodiments of the present invention may omit one or more of the method steps and/or perform the steps in a different order than the order listed. For example, some steps may not be performed in certain embodiments of the present invention. As a further example, certain steps may be performed in a different temporal order, including simultaneously, than listed above.

Certain embodiments include computer-readable media for carrying or having computer-executable instructions or data structures stored thereon. Such computer-readable media may be any available media that may be accessed by a general purpose or special purpose computer or other machine with a processor. By way of example, such computer-readable media may comprise RAM, ROM, PROM, EPROM, EEPROM, Flash, CD-ROM or other optical disk storage, magnetic disk storage or other magnetic storage devices, or any other medium which can be used to carry or store desired program code in the form of computer-executable instructions or data structures and which can be accessed by a general purpose or special purpose computer or other machine with a processor. Combinations of the above are also included within the scope of computer-readable media. Computer-executable instructions comprise, for example, instructions and data which cause a general purpose computer, special purpose computer, or special purpose processing machines to perform a certain function or group of functions.

Generally, computer-executable instructions include routines, programs, objects, components, data structures, etc., that perform particular tasks or implement particular abstract data types. Computer-executable instructions, associated data structures, and program modules represent examples of program code for executing steps of certain methods and systems disclosed herein. The particular sequence of such executable instructions or associated data structures represent examples of corresponding acts for implementing the functions described in such steps.

Embodiments of the present invention may be practiced in a networked environment using logical connections to one or more remote computers having processors. Logical connections may include a local area network (LAN), a wide area network (WAN), a wireless network, a cellular phone network, etc., that are presented here by way of example and not limitation. Such networking environments are commonplace in office-wide or enterprise-wide computer networks, intranets and the Internet and may use a wide variety of different communication protocols. Those skilled in the art will appreciate that such network computing environments will typically encompass many types of computer system configurations, including personal computers, hand-held devices, multi-processor systems, microprocessor-based or programmable consumer electronics, network PCs, minicomputers, mainframe computers, and the like. Embodiments of the invention may also be practiced in distributed computing environments where tasks are performed by local and remote processing devices that are linked (either by hardwired links, wireless links, or by a combination of hardwired or wireless links) through a communications network. In a distributed computing environment, program modules may be located in both local and remote memory storage devices.

An exemplary system for implementing the overall system or portions of embodiments of the invention might include a general purpose computing device in the form of a computer, including a processing unit, a system memory, and a system bus that couples various system components including the system memory to the processing unit. The system memory may include read only memory (ROM) and random access memory (RAM). The computer may also include a magnetic hard disk drive for reading from and writing to a magnetic hard disk, a magnetic disk drive for reading from or writing to a removable magnetic disk, and an optical disk drive for reading from or writing to a removable optical disk such as a CD ROM or other optical media. The drives and their associated computer-readable media provide nonvolatile storage of computer-executable instructions, data structures, program modules and other data for the computer.

While the invention has been described with reference to certain embodiments, it will be understood by those skilled in the art that various changes may be made and equivalents may be substituted without departing from the scope of the invention. In addition, many modifications may be made to adapt a particular situation or material to the teachings of the invention without departing from its scope. Therefore, it is intended that the invention not be limited to the particular embodiment disclosed, but that the invention will include all embodiments falling within the scope of the appended claims.

1. A computer-implemented method for automated determination of healthcare exam relevance and connectivity, said method comprising:
receiving an update message regarding a first exam record;
updating the first exam record based on the message;
matching one or more additional exam records to the first exam record based on one or more predefined exam attributes;
selecting one of the exam first record and the one or more additional exam records as an eligible exam record;
hiding display of the one or more exam records not selected as the eligible exam record;
displaying the eligible exam record;
receiving an additional update message for the first exam record;
evaluating the additional update message to determine applicability of the update message to the matching one or more additional exam records; and
selecting one or more of the first exam record and the matching one or more additional exam records as one or more eligible exam records based on evaluating the additional update message.

2. The method of claim 1, further comprising accepting user input regarding the one or more predefined exam attributes to set whether each of the one or more predefined exam attributes has optionality, a priority, or a weight.

3. The method of claim 1, further comprising generating one or more operational metrics for a healthcare workflow using the eligible exam record and a source system from which the exam record was retrieved.

4. The method of claim 3, wherein a plurality of eligible exam records are selected based on the additional update message and wherein the plurality of eligible exam records are used to generate the one or more operational metrics for the healthcare workflow.

5. The method of claim 1, wherein the evaluating the additional update message results in a switch from the first exam record as the eligible exam record to one of the matching one or more additional exam records as the eligible exam record.

6. The method of claim 1, further comprising evaluating a type of the additional update message to determine applicability of the additional update message to one or more of the first exam record and the matching one or more additional messages.

7. The method of claim 1, further comprising revising the matching of exam records based on the additional update message.

8. The method of claim 1, wherein the first exam record comprises a scheduled exam record and wherein one of the one or more additional exam records comprises an ordered exam record, and wherein the ordered exam record replaces the scheduled exam record as the eligible exam record based on the additional update message.

9. The method of claim 1, further comprising displaying the one or more eligible exam records on a dashboard indicating utilization and performance.

10. A tangible computer-readable storage medium having a set of instructions stored thereon which, when executed, instruct a processor to implement a method for automated determination of healthcare exam relevance and connectivity, said method comprising:
receiving an update message regarding a first exam record;
updating the first exam record based on the message;
matching one or more additional exam records to the first exam record based on one or more predefined exam attributes;
selecting one of the exam first record and the one or more additional exam records as an eligible exam record;
hiding display of the one or more exam records not selected as the eligible exam record;
displaying the eligible exam record;
receiving an additional update message for the first exam record;
evaluating the additional update message to determine applicability of the update message to the matching one or more additional exam records; and
selecting one or more of the first exam record and the matching one or more additional exam records as one or more eligible exam records based on evaluating the additional update message.

11. The computer-readable storage medium of claim 10, further comprising accepting user input regarding the one or more predefined exam attributes to set whether each of the one or more predefined exam attributes has optionality, a priority, or a weight.

12. The computer-readable storage medium of claim 10, further comprising generating one or more operational metrics for a healthcare workflow using the eligible exam record and a source system from which the exam record was retrieved.

13. The computer-readable storage medium of claim 12, wherein a plurality of eligible exam records are selected based on the additional update message and wherein the plurality of eligible exam records are used to generate the one or more operational metrics for the healthcare workflow.

14. The computer-readable storage medium of claim 10, wherein the evaluating the additional update message results in a switch from the first exam record as the eligible exam record to one of the matching one or more additional exam records as the eligible exam record.

15. The computer-readable storage medium of claim 10, further comprising evaluating a type of the additional update message to determine applicability of the additional update message to one or more of the first exam record and the matching one or more additional messages.

16. The computer-readable storage medium of claim 10, further comprising revising the matching of exam records based on the additional update message.

17. The computer-readable storage medium of claim 10, wherein the first exam record comprises a scheduled exam record and wherein one of the one or more additional exam records comprises an ordered exam record, and wherein the ordered exam record replaces the scheduled exam record as the eligible exam record based on the additional update message.

18. The computer-readable storage medium of claim 10, further comprising displaying the one or more eligible exam records on a dashboard indicating utilization and performance.

19. A healthcare system comprising:
a memory comprising one or more executable instructions and data;
a processor to execute the one or more executable instructions and to process the data; and
a user interface including a dashboard indicating utilization and performance metrics for a healthcare environment,
wherein the processor is to receive an update message regarding a first exam record and update the first exam record based on the message, the processor to match one or more additional exam records to the first exam record based on one or more predefined exam attributes and select one of the exam first record and the one or more additional exam records as an eligible exam record, wherein the processor is to hide display on the user interface of the one or more exam records not selected as the eligible exam record and displaying the eligible exam record via the user interface,
the processor to receive an additional update message for the first exam record, evaluate the additional update message to determine applicability of the update message to the matching one or more additional exam records, and select one or more of the first exam record and the matching one or more additional exam records as one or more eligible exam records based on evaluating the additional update message.

20. The system of claim 19, wherein the processor is to accept user input via the user interface regarding the one or more predefined exam attributes to set whether each of the one or more predefined exam attributes has optionality, a priority, or a weight.

21. The system of claim 19, wherein a plurality of eligible exam records are selected based on the additional update message and wherein the plurality of eligible exam records are used to generate the one or more operational metrics for the healthcare workflow.

22. The system of claim 19, wherein the processor evaluating the additional update message results in a switch from the first exam record as the eligible exam record to one of the matching one or more additional exam records as the eligible exam record.

23. The system of claim 19, wherein the processor is to evaluate a type of the additional update message to determine applicability of the additional update message to one or more of the first exam record and the matching one or more additional messages.

24. The system of claim 19, wherein the processor is to revise the matching of exam records based on the additional update message.

25. The system of claim 19, wherein the first exam record comprises a scheduled exam record and wherein one of the one or more additional exam records comprises an ordered exam record, and wherein the ordered exam record replaces the scheduled exam record as the eligible exam record based on the additional update message.
